(19) United States
(12) Patent Application Publication
(10) Pub. No.: US 2022/0275038 A1
(43) Pub. Date: Sep. 1, 2022

(54) CLSP DERIVATIVE INCAPABLE OF BEING AFFECTED BY CLSP INHIBITING SUBSTANCE, AND CLSP ACTIVITY ENHANCING/PROTECTING AGENT

(71) Applicant: TOKYO MEDICAL UNIVERSITY, Tokyo (JP)

(72) Inventors: Masaaki MATSUOKA, Tokyo (JP); Yuuichi HASHIMOTO, Tokyo (JP)

(73) Assignee: TOKYO MEDICAL UNIVERSITY, Tokyo (JP)

(21) Appl. No.: 17/630,311

(22) PCT Filed: Jul. 17, 2020
(86) PCT No.: PCT/JP2020/027764

§ 371 Date: Jan. 26, 2022

(30) Foreign Application Priority Data
Aug. 15, 2019 (JP) 2019-149216

(51) Int. Cl. C07K 14/47; C07K 14/575; A61P 25/28; G01N 33/50

(52) U.S. Cl. CPC C07K 14/4728; C07K 14/575; A61P 25/28; G01N 33/5058; C07K 2319/00

(57) ABSTRACT

[Problem] Provided is a calmodulin-like skin protein (CLSP) derivative, which has an activity to suppress neuronal cell dysfunction or cell death associated with e.g., Alzheitner's disease stronger than of humanin, and which is insensitive to an inhibitory action by an inhibitor of the activity; a polypeptide which has an action/activity to potentiate or protect the Alzheimer's disease-suppressing activity by CLSP; and the like.

[Solution] A derivative (mutant) of calmodulin-like skin protein (CLSP), characterized by including an endogenous humanin-homogenous region (EHR), which is the core of the activity to suppress the neuronal cell dysfunction or neuronal cell death associated with Alzheimer's disease (CLSP activity), and not including a region to which an inhibitor of the CLSP activity binds; a pharmaceutical composition to suppress the neuronal cell dysfunction or neuronal cell death associated with Alzheimer's disease, the composition including the mutant as an active ingredient; and the like.

### TECHNICAL FIELD

The present invention relates to a derivative of calmodulin-like skin protein (CLSP), which has an activity to suppress Alzheimer's disease (AD)-linked neuronal cell dysfunction or neuronal cell death, and is insensitive to an inhibitory or suppressive action by inhibiting substances (inhibitors) of the activity; a potentiator or protector of the activity by CLSP (also referred to as “AD-protecting activity,” “anti-AD activity,” “CLSP activity” or “cytotoxicity-suppressing activity by CLSP”), which consists of a polypeptide including the collagen-homologous region of adiponectin and the like; a fusion protein including e.g., CLSP or the CLSP derivative and the polypeptide; and a pharmaceutical composition including the above as an active ingredient, particularly a pharmaceutical composition for the treatment of Alzheimer's disease; and the like.

### BACKGROUND ART

Alzheimer's disease (AD) is a major neurodegenerative disease that causes dementia. The AD pathogenesis is still not fully clarified, and disease-modifying (disease-preventing and progression-suppressing) therapies for AD have been far from practical application (1-3).

The bioactive peptides, humanin and CLSP, are physiological agonists for the heterotrimeric humanin receptor (htHNR), consisting of ciliary neurotrophic factor receptor a, WSX-1, and gp130 (4-6). They inhibit AD-related neuronal cell death in vitro via htHNR (5, 7). The transgenic overexpression of CLSP protects against synaptic loss and memory loss in AD model mice (8). However, the activity of humanin is weak (50% effective concentration is 1 to 10 μM) (6, 7), and the in vivo concentration of humanin appears insufficient to exert the neuroprotective effect (6, 9).

CLSP is produced mainly in the skin keratinocytes and to a less extent, in epithelial cells of some peripheral tissues (10-12). Scopolamine-induced memory impairment in mice was ameliorated by intraperitoneal administration of CLSP (13). In addition, a sufficient amount of CLSP exists in human cerebrospinal fluids (14). From these experimental facts, CLSP is presumed to reach the central nervous system (CNS) from peripheral tissues via blood circulation and enter nerve tissues through the blood-brain barrier (14). EHR (endogenous humanin-homogenous region), which is a sequence consisting of 22 amino acids, 40-61, of CLSP, is essential for the CLSP activity (5) and the activity of wild-type CLSP is 10⁵-fold more potent than humanin (50% effective concentration is 10-100 pM) (5). From the measured concentration of CLSP in human cerebrospinal fluids (14), the concentration of CLSP in the CNS is estimated to be a concentration sufficient to exert the neuroprotective effect as an AD-protecting factor. From these published findings (5, 6, 8, 9, 13 and 14), CLSP, rather than humanin, is likely a main agonist for the htHNR in vivo. In addition, a previous study (35) has suggested that the activation levels of htHNR are reduced in the CNS of AD patients. Therefore, a possibility that the levels of CLSP, a main agonist for the htHNR, will be reduced in the CNS of AD patients was presented as further deduction. According to the latest study of the present inventors (14), however, the possibility that the CLSP levels themselves will be reduced in the CNS of AD patients was denied.

Humanin and CLSP, and their actions/effects are also described in detail in Patent Literature 1 in addition to references cited above with numbers.

Adiponectin is an adipose tissue-derived peptide hormone showing various metabolic actions such as an increase in insulin sensitivity, insulin-independent glucose uptake, and fatty acid degradation by binding to receptors such as adiponectin R1 and adiponectin R2 to activate AMP kinase-mediated intracellular signaling. Accordingly, this hormone is considered to play a role in suppressing type 2 diabetes, obesity, atherosclerosis, non-alcoholic fatty liver disease, and metabolic syndrome and associated metabolic abnormalities.

A plurality of studies has provided as preliminary data indirect evidence that the insufficiency of adiponectin or the abnormal regulation of adiponectin signaling is linked to the onset of AD as described below (31). An increase in serum adiponectin levels (29, 30) may be an independent risk factor of AD (32). Conversely, a study indicated that patients with type II diabetes, having a smaller concentration of serum adiponectin, developed AD-like pathology (33). Adiponectin levels are reduced in the CSF of AD patients and are inversely correlated with an increase in Aβ levels (30). Adiponectin knockout mice show AD-like symptoms and pathological findings (34).

### CITATION LIST

### Patent Literature

Patent Literature 1: Japanese Patent No. 5939528

### SUMMARY OF INVENTION

### Technical Problem

CLSP binds to a plurality of proteins in addition to htHNR (15); however, it remains unknown how the binding affects the function of CLSP.

A first subject of the present invention is to examine a possibility that these CLSP binding factors and CLSP binding factors newly developed in the present invention will regulate the CLSP activity and to analyze detailed mechanisms of proteins which regulate the activity.

A second subject is to verify that the CLSP activity is reduced in the central nervous system of AD using samples derived from AD patients and to examine a possibility that abnormalities of these CLSP binding factors will contribute to the onset of AD.

Furthermore, a third subject is to provide a CLSP derivative insensitive to the inhibitory or suppressive action by inhibitors of the CLSP activity, a potentiator or protector of the CLSP activity by CLSP and the CLSP derivative, a fusion protein of CLSP or the CLSP derivative and a potentiator or protector, and a pharmaceutical composition to suppress Alzheimer's disease-related neuronal cell dysfunction or neuronal cell death including the above as an active ingredient, and the like.

### Solution to Problem

As a result of diligent studies to solve the above subjects, the present inventors obtained findings described below first in the art, thereby completing the present invention.

First, it was found that the CLSP activity was suppressed by CLSP inhibiting substances (inhibitors) such as apolipoproteins E (ApoE; ApoE3 and ApoE4 used in this experiment are cognate proteins having one different amino acid and have almost the same biochemical properties), 14-3-3 proteins and calreticulin (FIG. 2 and FIG. 3). A series of data demonstrate that these CLSP inhibiting substances show full CLSP-suppressing effect at a concentration between equal to and 5-fold higher than the concentration of CLSP in media. It is known that a much higher concentration of ApoE than the concentration of CLSP exists in the human central nervous system. The concentration of ApoE in human cerebrospinal fluids (CSF), for example, is estimated to be 40-200 nM (18, 19), while the concentration of CLSP is estimated to be 3-6 nM (14). Therefore, given that the CLSP activity is provided by a simple system consisting of only CLSP and its inhibiting substance in the CNS in vivo, the activity of CLSP is considered to be completely nullified by such higher concentration of endogenous ApoE (in normal living bodies, CLSP-protecting substances exist as described below and keep the CLSP activity (FIG. 5, FIG. 6, FIG. 7). Therefore, in order that the CLSP activity reduced in the central nervous system of AD (FIGS. 10 and 11, Tables 1, 2, 3 and 4) will appear by increasing wild-type CLSP as a therapeutic means, the concentration of CLSP in the CNS needs to be increased to at least 40-200 nM or more to overcome the inhibition by CLSP-inhibiting substances. However, since CLSP cannot pass the blood-brain barrier efficiently to enter the central nervous system (5, 14), it is difficult to achieve this by administering wild-type CLSP via a peripheral route. In mice, for example, the concentrations of CLSP in the CSF and serum reach 5 nM and 500 nM respectively an hour (commonly expected to be the highest concentration) after intraperitoneal injection of 5 nmol wild-type CLSP (5). Therefore, the administration of at least about 10-fold or more the amount of wild-type CLSP is required to raise the concentration to 40-200 nM or more in the CSF by a simple calculation. However, 5 nmol administered in the above experiment is already very large to mice and it is realistically difficult to further increase the amount administered. That is, it is almost impossible that the CLSP activity will appear in the CNS by peripheral injection of wild-type CLSP. Therefore, in order that the CLSP activity will appear in the CNS by peripheral administration of CLSP, modifications or ideas to pass the blood-brain barrier more efficiently, and/or release the CLSP from the inhibitory effect by CLSP inhibiting substances are essential.

The present inventors found that ApoE4 bound to CLSP via the C-terminal region (amino acid 62-146) of CLSP (FIG. 9 and FIG. 15). This finding shows that the N-terminal region of CLSP (amino acid 1-61: abbreviated as “CLSP1-61”) does not bind to ApoE and is insensitive to ApoE-mediated suppression. Importantly, the present inventors further proved that CLSP1-61 had an activity equal to that of wild-type CLSP and suppressed V642I-APP-induced neuronal cell death (FIG. 18). In fact, the minimal concentrations of CLSP1-61 produced in E. coli and wild-type CLSP required to completely inhibit V642I-APP-induced neuron death are the same, 0.5 nM (FIG. 18 and FIG. 2).

As expected, the suppression of V642I-APP-induced neuronal cell death mediated by CLSP1-61 is not inhibited by not only ApoE3 but also other CLSP inhibitors such as 14-3-3σ protein or calreticulin (FIG. 19). The above demonstrated that CLSP1-61 was completely free from the suppression by CLSP inhibiting substances and also had almost the same activity as its wild-type and thus was a CLSP derivative showing the CLSP activity at concentration much lower than of the wild-type CLSP in vivo.

### [SEQ ID NO:1: CLSP (1-146)]

mageltpeeeaqykkafsavdtdgngtinaqelgaalkatgknlseaqlrklisevdsdgdgeisfqefltaakkaragledl qvafrafdqdgdghitvdelrramaglgqplpqeeldamireadvdqdgrvnyeefarmlaqe (although the 58^{th} “s” may be “g” due to genetic polymorphism, the activity is the same)

It was further found that adiponectin bound to the EHR (endogenous humanin-homogenous region) of CLSP (FIG. 1 and FIG. 9, FIG. 15) to potentiate the CLSP activity (activity-potentiating factor; FIG. 7) and protect (keep) the CLSP activity from all kinds of CLSP inhibiting substances (activity-protecting factor; FIG. 5, FIG. 6). In fact, even when the concentration of a CLSP inhibiting substance is very high, up to 50 nM, if 0.2-0.25 nM adiponectin exists, AD-related cell death is completely suppressed by 1 nM CLSP (FIG. 5 and FIG. 7). This result indicates that adiponectin is a CLSP activity-protecting factor that keeps the CLSP activity in the CNS, in which CLSP inhibiting substances exist at concentration much higher than that of CLSP.

Furthermore, an experiment using clinical samples found that adiponectin levels in the CSF were reduced to 0.3 nM in AD patients (FIG. 10, Tables 1 and 2). This result is consistent with the result of a previous study (30). It was further found that the intraneuronal CLSP signal intensity was reduced in AD patients (FIG. 11, Tables 3 and 4). Considering these results together, it is suggested that the adiponectin levels in the CNS are reduced due to some kind of cause in AD patients, and accordingly the CLSP activity is reduced so that neurons become sensitive to AD-related toxicity (i.e., neurotoxicity is exerted).

The present inventors further found that the collagen-homologous region of adiponectin (ADN) (ADNCol: corresponding to the amino acid 45-104 sequence in ADN) independently bound to CLSP (FIG. 15) and was sufficient to show the CLSP-potentiating/protecting activity (FIG. 20 and FIG. 21). The important fact is that the CLSP-potentiating/protecting activity of ADNCol is only slightly weaker than that of the wild-type adiponectin. In fact, the minimal concentration of wild-type adiponectin that imparts full CLSP-potentiating/protecting activity is 0.2-0.25 nM, while that of ADNCol is 0.5 nM. It is also known that a globular domain located at the C-terminal of adiponectin is essential to regulate the metabolic activity of adiponectin such as glucose-reducing effect via normal adiponectin receptors, AdipoR1 and 2 (42). Therefore, the globular domain-deficient ADNCol lacks these metabolic effects of adiponectin. That is, although ADNCol, which lacks the globular domain, has a full CLSP activity-potentiating/protecting action as is the case with the wild-type ADN, ADNCol cannot bind to the normal adiponectin receptors unlike the wild-type AND. Accordingly, ADNCol is not considered to show a so-called metabolism-regulating activity (an activity that can be an adverse effect). On the other hand, the previously published studies (33, 34) presume that the anti-AD activity of adiponectin is mediated by the metabolism-regulating activity induced by binding to the normal adiponectin receptors AdipoR1 and R2.

From the above matters, ADNCol as the CLSP potentiator/protector is expected to have four advantages over the wild-type adiponectin. First, given that the normal adiponectin receptors AdipoR1 and 2 (canonical adiponectin receptors) are rich in the in vivo tissues (CNS and peripheral tissues), a significant proportion of the wild-type adiponectin is used to produce complexes with the normal adiponectin receptors, but it is not presumed to be the case with ADNCol. Second, it is suggested that the levels of CSF adiponectin will be reduced in AD because it is used to form insoluble complexes with hyperphosphorylated tau in neurons (30), and it is presumed that this process is caused because the wild-type adiponectin binds to the normal adiponectin receptors and taken up by neurons. As ADNCol does not bind to the canonical adiponectin receptors, it is very likely that it will not form complexes with the hyperphosphorylated tau in neurons. The above two points suggest that the amount of ADNCol required to show the CLSP-potentiating/protecting activity in vivo can be smaller than that of the wild-type adiponectin. Third, a large amount of the wild-type adiponectin may cause adverse effects by binding to the normal adiponectin receptors to activate various metabolic pathways. However, it is presumed that ADNCol does not bind to the normal receptors and thus does not cause the adverse effects. Fourth, compared to the amino acid length of the wild-type adiponectin (244 amino acids: SEQ ID NO:3), the amino acid length of ADNCol (60 amino acids: SEQ ID NO:2) is relatively short and thus is easy to industrially produce. Because of all these advantages of ADNCol, ADNCol is superior to the wild-type adiponectin as an anti-AD agent.

The present inventors also found that a fusion protein (hybrid peptide) of CLSP or the CLSP derivative and the above potentiator or protector had more potent protective activity against the V642I-APP-induced neuronal cell death than CLSP1-61 and the wild-type CLSP (FIG. 22). That is, the minimal concentrations of the hybrid peptide consisting of CLSP1-61 and ADNCol (named “CLSPCOL”) and the hybrid peptide consisting of the wild-type CLSP and ADNCol (named “wt-CLSPCOL”) that completely suppressed V642I-APP-induced neuronal cell death were 0.1 nM and the minimal concentrations of CLSP1-61 and the wild-type CLSP were 0.5 nM (FIG. 22). In addition, CLSPCOL and the wt-CLSPCOL are not suppressed by the CLSP inhibitors or, they are suppressed mildly (FIGS. 24 and 25).

It was further found that CLSPCOL penetrated the blood-brain barrier and was transferred to the CNS more efficiently than wt-CLSPCOL (FIG. 23 and Table 1). That is, the concentrations of CLSPCOL an hour after intraperitoneal injection of 10 nmol CLSPCOL in mice were 72 nM in interstitial fluid (ISF)-containing brain homogenates and 320 nM in serum (FIG. 23 and Table L1).

### [ADNCol: SEQ ID NO:2]

ghpghngapgrdgrdgtpgekgekgdpgligpkgdigetgvpgaegprgfpgiqgrkgep

### [ADN: SEQ ID NO:3]

mlllgavllllalpghdqetttqgpgvllplpkgactgwmagipghpghngapgrdgrdgtpgekgekgdpgligpkgd igetgvpgaegprgfpgiqgrkgepgegayvyrsafsvgletyvtipnmpirftkifynqqnhydgstgkfhcnipglyyf ayhitvymkdvkvslfkkdkamlftydqyqennvdqasgsvllhlevgdqvwlqvygegernglyadndndstftgfl lyhdtn

That is, the present invention relates to the following aspects.

### [Aspect 1]

A derivative (mutant) of calmodulin-like skin protein (CLSP), including an endogenous humanin-homogenous region (EHR), which is the core of an activity to suppress Alzheimer's disease-related neuronal cell dysfunction or neuronal cell death (CLSP activity), and not including a region to which an inhibitor of the CLSP activity binds.

### [Aspect 2]

The derivative according to aspect 1, wherein EHR consists of an amino acid sequence (I):
TGKNLSEAQLRKLISEVDS(or “G”)DGD (amino acid single letter code) (I).

### [Aspect 3]

The derivative according to aspect 1 or 2, wherein the region to which the inhibitor binds is the C-terminal amino acid sequence region (amino acid 62-146) of CLSP (SEQ ID NO:1).

### [Aspect 4]

The derivative according to any one of aspects 1 to 3, which is a polypeptide consisting of an amino acid sequence below:

(1) an N-terminal amino acid sequence region (amino acid 1-61) of CLSP;

(2) an amino acid sequence of the (1) above, wherein one or several (e.g., about 2-5) amino acids are deleted, substituted or inserted in an amino acid sequence other than EHR included in the amino acid sequence of the (1) above; or

(3) an amino acid sequence of the (1) above, which has an identity of 90% or more, preferably 95% or more, and further preferably 98% or more to an amino acid sequence other than EHR included in the amino acid sequence of the (1) above.

### [Aspect 5]

The derivative according to any one of aspects 1 to 4, which is insensitive to an inhibitory or suppressive action by the inhibitor of the CLSP activity.

### [Aspect 6]

The derivative according to any one of aspects 1 to 5, wherein the inhibitor is selected from the group consisting of apolipoprotein E, 14-3-3 proteins, and calreticulin.

### [Aspect 7]

A polypeptide consisting of an amino acid sequence below:

(1) an amino acid sequence (ADNCol) shown by SEQ ID NO:2;

(2) an amino acid sequence including the amino acid sequence (ADNCol) of the (1) above;

(3) an amino acid sequence of adiponectin shown by SEQ ID NO:3, wherein one or several amino acids are deleted, substituted or inserted in an amino acid sequence other than ADNCol included in the amino acid sequence shown by SEQ ID NO:3; or

(4) an amino acid sequence of adiponectin shown by SEQ ID NO:3, which has an identity of 90% or more to an amino acid sequence other than ADNCol included in the amino acid sequence shown by SEQ ID NO:3.

### [Aspect 8]

A potentiator or protector of the CLSP activity by CLSP or the CLSP derivative according to aspect 1, which consists of a polypeptide according to aspect 7.

### [Aspect 9]

The potentiator or protector according to aspect 8, which protects the CLSP from the inhibitory or suppressive action by the inhibitor of the CLSP activity, or nullifies the action by the inhibitor.

### [Aspect 10]

The potentiator or protector according to aspect 8 or 9, wherein the polypeptide is adiponectin.

### [Aspect 11]

The potentiator or protector according to any one of aspect 8 to 10, wherein the inhibitor is selected from the group consisting of apolipoprotein E, 14-3-3 proteins, and calreticulin.

### [Aspect 12]

A fusion protein including CLSP or the CLSP derivative according to claim 1 and the polypeptide according aspect 7.

### [Aspect 13]

The fusion protein according to aspect 12, consisting of the N-terminal amino acid sequence region (amino acid 1-61) of CLSP, and ADNCol.

### [Aspect 14]

The fusion protein according to aspect 12 or 13, which is insensitive to the inhibitory or suppressive action by the inhibitor of the CLSP activity.

### [Aspect 15]

A pharmaceutical composition to suppress Alzheimer's disease-related neuronal cell dysfunction or neuronal cell death, the composition including as an active ingredient the CLSP derivative according to any one of aspects 1 to 6, the polypeptide according to aspect 7, the potentiator or protector according to any one of aspects 8 to 11, or the fusion protein according to any one of aspects 12 to 14.

[Aspect 16]

The pharmaceutical composition according to aspect 15, which is used to prevent or treat diseases accompanied by Alzheimer's disease-related memory impairment or neurodegeneration.

### [Aspect 17]

A method for treating a disorder accompanied by neuronal cell dysfunction or neuronal cell death, or a disease accompanied by memory impairment or neurodegeneration, the method including a stage of administering the pharmaceutical composition according to aspect 15 or 16 to an individual affected with or suspected of the disorder or the disease.

### [Aspect 18]

The method according to aspect 17, wherein the disorder or disease is Alzheimer's disease.

### [Aspect 19]

A method for detecting an activity to suppress Alzheimer's disease-related neuronal cell dysfunction or neuronal cell death by the CLSP derivative according to any one of aspects 1 to 6, the polypeptide according to aspect 7, or the potentiator or protector according to any one of aspects 8 to 11, or the fusion protein according to any one of aspects 12 to 14 (collectively referred to as “the polypeptide of the invention”), the method including (a) the step of inducing the neuronal cell dysfunction or neuronal cell death in the presence/absence of the CLSP inhibitor, and in the presence/absence of the polypeptide of the invention, (b) the step of detecting the neuronal cell dysfunction or neuronal cell death, and (c) the step of comparing the neuronal cell dysfunction or neuronal cell death in the presence/absence of the polypeptide of the invention.

### [Aspect 20]

A method for screening a substance regulating an activity to suppress Alzheimer's disease-related neuronal cell dysfunction or neuronal cell death by the CLSP derivative according to any one of aspects 1 to 6, the polypeptide according to aspect 7, or the potentiator or protector according to any one of aspects 8 to 11, or the fusion protein according to any one of aspects 12 to 14 (collectively referred to as “the polypeptide of the invention”), or CLSP,

the method including (a) the step of inducing the neuronal cell dysfunction or neuronal cell death with or without a test substance in the presence of the polypeptide of the invention or CLSP, (b) the step of detecting the neuronal cell dysfunction or neuronal cell death, and (c) the step of selecting a substance regulating the activity to suppress the neuronal cell dysfunction or neuronal cell death by the polypeptide of the invention or CLSP. Advantageous Effect of Invention

The CLSP derivative of the present invention includes an endogenous humanin-homogenous region (EHR), which is the core of an activity to suppress Alzheimer's disease-related neuronal cell dysfunction or neuronal cell death (CLSP activity), and does not include a region to which an inhibitor of the CLSP activity, such as ApoE, or 14-3-3σ protein or calreticulin, binds.

Consequently, the CLSP derivative has the CLSP activity equivalent to that of wild-type CLSP and is substantially (significantly) insensitive to the inhibitory or suppressive action by the inhibitors of the CLSP activity. From the above, these polypeptides are completely free from the inhibition and suppression by the CLSP inhibitors and show the CLSP activity at much lower concentrations than of wild-type CLSP in vivo.

A polypeptide which consists of the amino acid sequence shown by SEQ ID NO: 2 and is the collagen-homologous region of adiponectin, and a polypeptide including the amino acid sequence shown by SEQ ID NO:2, for example multimeric, such as trimeric, adiponectin, bind to the EHR in CLSP and CLSP1-61 in the CLSP derivative of the present invention, and have an action/effect to potentiate the CLSP activity thereof.

The above polypeptides further have an action/effect to protect the CLSP from the inhibition or suppression by the inhibitors of the CLSP activity such as apolipoprotein E or nullify the inhibitory or suppressive action by the inhibitors. Therefore, the above polypeptides are useful as a potentiator or protector of the activity to suppress Alzheimer's disease-related neuronal cell dysfunction or neuronal cell death.

In addition, the fusion protein of the present invention has more potent anti-AD activity than of a derivative consisting of CLSP or a part of CLSP. The fusion protein is also insensitive to the inhibition by the CLSP inhibitors or, even when it is affected by the inhibition, the degree is very moderate. Furthermore, the peptide is expected to lack metabolic activity derived from adiponectin and also not to be used to form complexes with canonical adiponectin receptors. In addition to these advantages, CLSPCOL, one of the fusion proteins, has a feature that the transfer through the blood-brain barrier is extremely good and thus can be likely an ideal anti-AD agent which can be peripherally administered.

### BRIEF DESCRIPTION OF DRAWINGS

FIG. 1 shows apolipoproteins E3 and E4, and adiponectin bind to CLSP. Apolipoproteins E3 and E4, adiponectin, and annexin II, C-terminally tagged with HA, were overexpressed in F11 neurohybrid cells by transfection. At 24 hours after the transfection, the F11 cells were harvested for the preparation of cell lysates. A suitable amount of GST-MycHis or CLSP-MycHis-conjugated sepharose 4B separately adjusted was added to 300 μg of lysates, incubated at 4° C. overnight and exhaustively washed, followed by pull-down precipitation. Inputs including the cell lysates and the sepharose 4B beads conjugating GST-MycHis (GST-MH) and CLSP-MycHis (CLSP-MH), and the pulled-down precipitates of cell lysates were subjected to SDS-PAGE and then immunoblot analysis using HA (hemagglutinin A) and myc antibodies.

FIG. 2 shows apolipoproteins E3 and E4 suppress the CLSP activity. (a) SH-SY5Y cells were transfected with the pcDNA3.1/MycHis vector (vector) or pcDNA3.2/MycHis-V642I-APP (V642I-APP). The cells were then cultured in DMEM/F12-10% FBS containing indicated concentrations of CLSP-MycHis. At 24 hours after the transfection, the media were replaced with DMEM/F12 with N2 supplement containing the same concentration of CLSP-MycHis. At 48 hours after the onset of the transfection, the cells were subjected to cell viability assays using the WST-8 cell death assay kit, or staining with calcein AM, and trypan blue exclusion cell mortality assays. The cell lysates were also immunoblotted using the APP antibody, 22C11. (b, c) SH-SY5Y cells were transfected with the pcDNA3.1/MycHis vector (vector) or pcDNA3.1/MycHis-V642I-APP (V642I-APP). The cells were then cultured in DMEM/F12-10% FBS containing 1 nm GST-MycHis or CLSP-MycHis with/without indicated concentrations of BSA, apolipoprotein E3 (b), or E4 (c). At 24 hours after the transfection, the media were replaced with DMEM/F12 with N2 supplement containing 1 nM GST-MycHis or GST-MycHis with/without the same concentration of BSA, apolipoprotein E3 (b) or E4 (c). At 48 hours after the onset of the transfection, the cells were harvested to perform the trypan blue exclusion cell mortality assays. The cell lysates were also immunoblotted using the APP antibody, 22C11.

FIG. 3 shows 14-3-3 family proteins and secreted calreticulin suppress the CLSP activity. (a-e) SH-SY5Y cells were transfected with the pcDNA3.1/MycHis vector (vector) or pcDNA3.1/MycHis-V642I-APP (V642I-APP). The cells were then cultured in DMEM/F12-10% FBS containing 10 nM GST-MycHis or CLSP-MycHis with/without indicated concentrations of BSA and a 14-3-3 isoform. At 24 hours after the transfection, the media were replaced with DMEM/F12 with N2 supplement containing 10 nM GST-MycHis or CLSP-MycHis with/without the same concentration of BSA or the 14-3-3 isoform. At 48 hours after the onset of the transfection, the cells were harvested to perform the trypan blue exclusion cell mortality assays. (f) SH-SY5Y cells were transfected with the empty pcDNA3.1/MycHis vector (vector) or pcDNA3.1/MycHis-V642I-APP (V642I-APP). The cells were then cultured in DMEM/F12-10% FBS containing 10 nM GST-MycHis or CLSP-MycHis with/without 10 nM BSA, calreticulin, annexin II, or annexin V At 24 hours after the transfection, the media were replaced with DMEM/F12 with N2 supplement containing the same concentration of GST-MycHis or CLSP-MycHis with/without 10 nM BSA, calreticulin, annexin II, or annexin V. At 48 hours after the onset of the transfection, the cells were harvested to perform the trypan blue exclusion cell mortality assays. The cell lysates were immunoblotted using the APP antibody, 22C11.

FIG. 4 shows adiponectin protects the CLSP activity from the inhibition by apolipoprotein E3. (a-c) SH-SY5Y cells were transfected with the empty pcDNA3.1/MycHis vector (vector) or pcDNA3.1/MycHis-V642I-APP (V642I-APP). The cells were then cultured in DMEM/F12-10% FBS containing 1 nM GST-MycHis or CLSP-MycHis with/without 10 nM adiponectin (a), annexin II (b), or annexin V (c). At 24 hours after the transfection, the media were replaced with DMEM/F12 with N2 supplement containing the same combination of proteins. At 48 hours after the onset of the transfection, the trypan blue exclusion cell mortality assays were performed. The cell lysates were also immunoblotted using the APP antibody, 22C11.

FIG. 5 shows adiponectin protects the CLSP activity from the inhibition by apolipoprotein E4. (a) SH-SY5Y cells were transfected with the pcDNA3.1/MycHis vector (vector) or pcDNA3.1/MycHis-V642I-APP/MycHis-V642I-APP (V642I-APP). The cells were then cultured in DMEM/F12-10% FBS containing 1 nM GST-MycHis or CLSP-MycHis with/without 10 nM apolipoprotein E4 with/without indicated concentrations of adiponectin. At 24 hours after the transfection, the media were replaced with DMEM/F12 with N2 supplement containing the same combination of proteins. At 48 hours after the onset of the transfection, the cells were harvested to perform the WST-8 and trypan blue exclusion cell mortality assays. The cell lysates were immunoblotted using the APP antibody, 22C11. (b) SH-SY5Y cells were transfected with the pcDNA3.1/MycHis vector (vector) or pcDNA3.1/MycHis-V642I-APP (V642I-APP). The cells were then cultured in DMEM/F12-10% FBS containing 1 nM GST-MycHis or CLSP-MycHis with/without stepwise increasing concentrations of apolipoprotein E4 with/without 1 nM adiponectin. At 24 hours after transfection, the media were replaced with DMEM/F12 with N2 supplement containing the same combination of proteins. At 48 hours after the onset of the transfection, the cells were harvested to perform the WST-8 and trypan blue exclusion cell mortality assays. The cell lysates were immunoblotted using the APP antibody, 22C11.

FIG. 6 shows adiponectin protects CLSP from the inhibition by 14-3-3σ and calreticulin. (a, b) SH-SY5Y cells were transfected with the pcDNA3 vector (vector) or pcDNA3-V642I-APP (V642I-APP). The cells were then cultured in DMEM/F12-10% FBS containing 1 nM GST-MycHis or CLSP-MycHis with/without 1 nM adiponectin with/without 2 nM 14-3-3σ (a) or 10 nM calreticulin (b). At 24 hours after the transfection, the media were replaced with DMEM/F12 with N2 supplement containing the same combination of proteins. At 48 hours after the onset of the transfection, the cells were harvested to perform the trypan blue exclusion cell mortality and WST-8 assays. The cell lysates were immunoblotted using the APP antibody, 22C11.

FIG. 7 shows adiponectin potentiates the CLSP activity. (a) SH-SY5Y cells were transfected with the pcDNA3.1/MycHis vector (vector) or pcDNA3.1/MycHis-V642I-APP (V642I-APP). The cells were then cultured in DMEM/F12-10% FBS containing indicated concentrations of GST-MycHis or CLSP-MycHis in culture fluid with/without 200 pM adiponectin. At 24 hours after the transfection, the media were replaced with DMEM/F12 with N2 supplement containing the same combination of proteins. (b) SH-SY5Y cells were transfected with the pcDNA3.1/MycHis vector (vector) or pcDNA3.1/MycHis-V642I-APP (V642I-APP). The cells were then cultured in DMEM/F12-10% FBS with indicated concentrations of GST-MycHis or CLSP-MycHis with/without indicated concentrations of adiponectin. At 24 hours after the transfection, the media were replaced with DMEM/F12 with N2 supplement containing the same combination of proteins. At 48 hours after the onset of the transfection, the cells were harvested to perform the trypan blue exclusion cell mortality and WST-8 assays. The cell lysates were immunoblotted using the APP antibody, 22C11.

FIG. 8 shows the dissociation constant for the binding between adiponectin and CLSP approximates the dissociation constant for the binding between apolipoprotein E4 and CLSP. (a) The presence of adiponectin slightly suppresses the binding between apolipoprotein E4 and CLSP. PBS containing CLSP-MycHis-conjugated sepharose 4B was mixed with any one or two of adiponectin, annexin II, recombinant apolipoprotein E3 or E4 and incubated at 4° C. overnight, followed by exhaustive washing. The estimated final concentration of each recombinant protein in the assays was 1 nM. The precipitates produced by pull-down, and the CLSP-MycHis-conjugated sepharose 4B beads and each recombinant protein as inputs were then subjected to SDS-PAGE, followed by visualization with silver staining. (b) Scatchard analysis was performed to measure dissociation constants. Each well in the 96-well plates coated with 20 pM recombinant apolipoprotein E4 or adiponectin was filled with stepwise increasing concentrations of CLSP-HiBiT and incubated at room temperature for 2 hours, followed by the estimation of HiBiT activity by measurement of chemiluminescence using Wallac ARVO™ X5 (Perkin Elmer). This experiment was performed in N=2, and mean data of two wells (Mean) were used for further analysis. From each mean CLSP-HiBiT activity (Mean), the mean CLSP-HiBiT activity at the zero concentration (background) was subtracted to give the real CLSP-HiBiT activity that was bound to adiponectin or apolipoprotein E4 (Del/MEAN). Then, the concentration of CLSP-HiBiT that was bound to apolipoprotein E4 or adiponectin (shown as <B>) was estimated, referring to a standard dose-response curve constructed based on the concentrations of CLSP-HiBiT and the corresponding chemiluminescence intensities (i.e., CLSP-HiBiT activity). Then, the concentration of free CLSP-HiBiT (unbound concentration) (shown as <F>) and B/F were calculated. Dissociation constants were calculated by the Scatchard analysis using Prism7 software.

FIG. 9 shows apolipoprotein and adiponectin bind to different sites of CLSP. (a) A schematic illustration of deletion mutants of CLSP was shown. (b) Apolipoprotein E4 (ApoE4) and adiponectin (ADN), C-terminally tagged with FLAG, were overexpressed in F11 neurohybrid cells by transfection. At 24 hours after the transfection, the F11 cells were harvested for the preparation of cell lysates. For the immunoprecipitation of ApoE4-FLAG and ADN-FLAG using FLAG antibody, 300 μg of cell lysates was used. Separately, recombinant CLSP-MycHis (FL-MH) or C-terminally MycHis-tagged CLSP deletion mutants produced in bacteria were purified. These immunoprecipitates and recombinant proteins as inputs were then subjected to SDS-PAGE and immunoblot analysis using the myc and FLAG antibodies. (C) The ApoE4-FLAG and ADN-FLAG immunoprecipitates produced in (b) were mixed with purified recombinant CLSP-MycHis (FL-MH) or C-terminally MycHis-tagged CLSP deletion mutants and incubated at 4° C. overnight, followed by exhaustive washing. The pulled-down precipitates were then subjected to SDS-PAGE and immunoblot analysis using the myc and FLAG antibodies.

FIG. 10 shows adiponectin is reduced in the CSF of AD patients. (a) The concentrations of CSF adiponectin in AD patients and non-AD control shown in Table 1 were measured using an adiponectin ELISA system. Stepwise increasing concentrations of recombinant adiponectin were measured to create a standard dose-response line. (b) The concentrations of CSF adiponectin in AD patients and non-AD control were plotted as dots (N=14 for AD cases, N=20 for non-AD cases). The mean±SEM concentrations of adiponectin are also shown (AD, 0.31±0.13 nM; non-AD, 0.96±0.19 nM; unpaired t-test, p=0.0065). (c) The concentrations of CSF adiponectin in AD patients and non-AD control aged 81-88 years were plotted as dots (N=6 for AD cases, N=5 for non-AD cases). The mean±SEM concentrations of adiponectin are also shown (AD, 0.30±0.07 nM; non-AD, 1.41±0.16 nM; unpaired t-test, p<0.0001).

FIG. 11 shows the levels of intraneuronal SH3BP5 were reduced in AD cortices. (a) Outer pyramidal layers of temporal or occipital lobes from two AD patients (65-year-old, male; 79-year-old, female) and ALS patients (66-year-old, male; 79-year-old, male) were immunostained with the antibody to SH3BP5. Immunodetection was performed by Tyramide-Red method. Scale bars, 200 mm. (b) Example of quantification of cell immunofluorescence intensity. The cell area and the non-cell area surrounding the cell were surrounded by marks and mean immunofluorescence intensity of the cell area (x) and mean immunofluorescence intensity of the non-cell area surrounding the cell (y) were measured. The relative mean immunofluorescence intensity in the neuron was then calculated by (x-y), and the x-y value was multiplied by the neuronal area to calculate the levels of SH3BP5 expression in the neuron. (c) Sections of outer pyramidal layers of temporal or occipital lobes from AD patients and patients with amyotrophic lateral sclerosis (ALS) (including those shown in (a)) were immunostained with the antibody to SH3BP5 as shown in Table 2. Immunodetection was performed by Tyramide-Red method. As described in “Materials and methods” in detail, the immunofluorescence intensity was measured using Image J 1.37v. The relative intensities in AD patients and ALS patients were plotted as dots (N=7 for AD cases, N=6 for ALS cases). The means±SEM of immunofluorescence intensities were also shown (AD, 46564±7737 arbitrary unit; ALS, 79225±10305 arbitrary unit; unpaired t-test, p=0.0256). (d, e) The concentrations of SH3BP5 in 20 μL of lysates obtained from temporal cortices of AD and non-AD patients were measured using SH3BP5 ELISA as shown in Table 3. Stepwise increasing concentrations of recombinant SH3BP5 were measured to create a standard dose-response line (d). The relative concentrations of SH3BP5 in AD and non-AD patients were plotted (N=10 for each group) (e). The means±SD of relative SH3BP5 levels were also shown (AD, 103.9±9.0 arbitrary unit; normal, 159.4±16.5 arbitrary unit; unpaired t-test, p=0.0084).

FIG. 12 shows adiponectin itself does not inhibit the V642I-APP-induced neuronal cell death and not inhibit the CLSP-mediated reduction of V642I-APP-induced neuronal cell death. SH-SYSY cells were transfected with the pcDNA3.1/MycHis vector (vector) or pcDNA3.1/MycHis-V642I-APP (V642I-APP). The cells were then cultured in DMEM/F12-10% FBS with GST-MH or CLSP-MH with/without stepwise increasing concentrations of adiponectin. At 24 hours after the transfection, the media were replaced with DMEM/F12 with N2 supplement with GST-MH or CLSP-MH with/without stepwise increasing concentrations of adiponectin. At 48 hours after the onset of the transfection, the cells were harvested to perform the cell viability assays using the WST-8 cell death assay kit (Dojindo, Kumamoto, Japan) or staining with calcein AM (Dojindo), and the trypan blue exclusion cell mortality assays. In addition, the cell lysates were immunoblotted using the APP antibody, 22C11.

FIG. 13 shows 14-3-3σ levels in human CSF are not more than the detection limit. (a) The concentrations of 14-3-3σ in 20 μL of CSF from 8 non-AD patients (CSF #1-8) were measured using a 14-3-3σ ELISA system. The experiment was performed twice. Raw measured numbers for stepwise increasing concentrations of standard 14-3-3σ (concentrations; 0.195 to 6.25 nM) and the CSF of 8 non-AD patients were shown in Abs450 columns. Means of two values were then calculated and shown in Mean Abs450 columns. PBS was used as the negative control. Del Abs 450 nm numbers were obtained by subtracting the PBS number from each mean number. (b) Stepwise increasing concentrations of recombinant 14-3-3σ were measured to create a standard dose-response line. Thus, the lower limit detectable by this ELISA was estimated to be 0.4 nM. The concentrations of CSF 14-3-3σ in Del Ab 450 nM data in (a) are not more than the detection limit.

FIG. 14 shows trimeric adiponectin has CLSP-potentiating effect comparable to that of wild-type adiponectin. SH-SYSY cells were transfected with the pcDNA3.1/MycHis vector (vector) or pcDNA3.1/MycHis-V642I-APP (V642I-APP). The cells were then cultured in DMEM/F12-10% FBS containing indicated concentrations of GST-MycHis or CLSP-MycHis with/without 1 nM trimeric or wild-type (mono) adiponectin. At 24 hours after the transfection, the media were replaced with DMEM/F12 with N2 supplement containing the same combination of proteins. At 48 hours after the onset of the transfection, the cells were harvested to perform the WST-8 assays and trypan blue exclusion cell mortality assays. The cell lysates were immunoblotted using the APP antibody, 22C11. “***” p<0.001; “n.s.” no significance.

FIG. 15 shows detailed analysis of the binding between CLSP and ApoE4 or adiponectin. (a, b) <Apolipoprotein E4 binds to the C-terminal region of CLSP> A schematic illustration of deletion mutants of CLSP is shown in (a). Apolipoprotein E4 (ApoE4) and adiponectin (ADN), C-terminally tagged with FLAG, were overexpressed in F11 neurohybrid cells by transfection. At 24 hours after the transfection, the cell lysates of F11 cells were prepared. For immunoprecipitation of ApoE4-FLAG and ADN-FLAG using the FLAG antibody, 300 μg of cell lysates was used. Recombinant CLSP-MycHis (FL-MH) or C-terminally MycHis-tagged CLSP deletion mutants produced in bacteria were purified. These immunoprecipitates and recombinant proteins were then subjected to SDS-PAGE and immunoblot analysis using the myc and FLAG antibodies (inputs). The ApoE4-FLAG and ADN-FLAG immunoprecipitates were mixed with recombinant CLSP-MycHis (FL-MH) or C-terminally MycHis-tagged CLSP deletion mutants and incubated at 4° C. overnight, followed by exhaustive washing. The pulled-down precipitates were then subjected to SDS-PAGE and immunoblotted using the myc and FLAG antibodies. (c) <CLSP binds to the collagen-homologous region of adiponectin> The collagen-homologous region of adiponectin (ADNCol), N-terminally tagged with 6×His and G(HisG), was produced in bacteria. In addition, CLSP-FLAG was overexpressed in F11 neurohybrid cells by transfection. HisG-ADNCol, and purified recombinant FLAG-CLSP and a control (vector) immunoprecipitated with the FLAG antibody were subjected to SDS-PAGE and immunoblot analysis with the FLAG and HisG antibodies (inputs; left panel). Purified recombinant HisG-ANDCol and the immunoprecipitated CLSP-FLAG or control (vector) were then mixed and incubated at 4° C. overnight, followed by exhaustive washing. The pulled-down precipitates were then subjected to SDS-PAGE and immunoblot analysis using the FLAG and HisG antibodies (Co-IP; right panel).

FIG. 16 shows no correlation between age and CSF adiponectin concentration. Raw data of adiponectin levels and ages were plotted in all subjects in Table 1 and Table S1 (X-axis: age; Y-axis: CSF adiponectin concentration). The correlation coefficient is 0.0055.

FIG. 17 shows SH3BP5 levels in neurons are not affected by aging. SH3BP5 data of AD patients or ALS patients shown in FIG. 11C were divided into two groups based on the age and compared. One group is composed of persons with an age of 70 or less and the other with an age of 71 or more. Mean±SEM relative intensities of SH3BP5 in two groups were 57439±14465 arbitrary unit and 65237±7976 arbitrary unit (unpaired t-test, p=0.6328, t=0.49, R-squared=0.021, degree of freedom=11, p value by F-test=0.24).

FIG. 18 shows the minimal concentration of CLSP1-61 that completely suppresses V642I-APP-induced neuronal cell death is 500 pM. (a, b) SH-SY5Y cells were transfected with the pcDNA3.1/MycHis vector (vector) or pcDNA3.1/MycHis-V642I-APP (V642I-APP). The cells were then cultured in DMEM/F12-10% FBS containing indicated concentrations of GST-MycHis or CLSP(1-61)-MycHis. At 24 hours after the transfection, the media were replaced with DMEM/F12 with N2 supplement containing the same concentration of GST-MycHis or CLSP(1-61)-MycHis. At 48 hours after the onset of the transfection, the cells were harvested to perform the trypan blue exclusion cell mortality, WST8 and calcein assays. The cell lysates were also immunoblotted using the APP antibody, 22C11.

FIG. 19 shows CLSP inhibiting substances do not inhibit the CLSP1-61-mediated suppressive effect on V642I-APP-induced neuronal cell death. SH-SY5Y cells were transfected with the pcDNA3.1/MycHis vector (vector) or pcDNA3.1/MycHis-V642I-APP (V642I-APP). The cells were then cultured in DMEM/F12-10% FBS containing 1 nM GST-MycHis or CLSP(1-61)-MycHis with 10 nM BSA, ApoE3, 14-3-3σ, or calreticulin. At 24 hours after the transfection, the media were replaced with DMEM/F12 with N2 supplement containing GST-MycHis or CLSP(1-61)-MycHis with the same concentration of BSA, ApoE3, 14-3-3σ, or calreticulin. At 48 hours after the onset of the transfection, the cells were harvested to perform the trypan blue exclusion cell mortality, WST8, and calcein assays. The cell lysates were immunoblotted using the APP antibody, 22C11.

FIG. 20 shows the collagen-homologous region of adiponectin potentiates the CLSP activity. SH-SY5Y cells were transfected with the pcDNA3.1/MycHis vector (vector) or pcDNA3.1/MycHis-V642I-APP (V642I-APP). The cells were then cultured in DMEM/F12-10% FBS containing 1 nM or 50 pM GST-MycHis or CLSP-MycHis with 1 nM BSA, adiponectin (FL) or the collagen-homologous region (Col) of adiponectin. At 24 hours after the transfection, the media were replaced with DMEM/F12 with N2 supplement containing the same combination of proteins. At 48 hours after the onset of the transfection, the cells were harvested to perform the trypan blue exclusion cell mortality, WST8, and calcein assays. The cell lysates were immunoblotted using the APP antibody, 22C11.

FIG. 21 shows the minimal concentration of the collagen-homologous region of adiponectin that enables 50 pM CLSP to be fully active is 500 pM. SH-SYSY cells were transfected with the pcDNA3.1/MycHis vector (vector) or pcDNA3.1/MycHis-V642I-APP (V642I-APP). The cells were then cultured in DMEM/F12-10% FBS containing 50 pM GST-MycHis or CLSP-MycHis with 500 μM BSA, 250 μM adiponectin (FL) or indicated concentrations of the collagen-homologous region (Col) of adiponectin. At 24 hours after the transfection, the media were replaced with DMEM/F12 with N2 supplement containing the same combination of proteins. At 48 hours after the onset of the transfection, the cells were harvested to perform the trypan blue exclusion cell mortality, WST8 and calcein assays. The cell lysates were immunoblotted using the APP antibody, 22C11.

FIG. 22 shows CLSPCOL has potent AD-protecting activity. SH-SYSY cells were transfected with the pcDNA3.1/MycHis vector (vector) or pcDNA3.1/MycHis-V642I-APP (V642I-APP). The cells were then cultured in DMEM/F12-10% FBS containing 1 nM GST-MycHis, CLSP1-61-MycHis, CLSP-MycHis, or indicated concentrations of CLSPCOL or wt-CLSPCOL. At 24 hours after the transfection, the media were replaced with DMEM/F12 with N2 supplement containing the same concentration of reagents. At 48 hours after the onset of the transfection, the cells were harvested to perform the trypan blue exclusion cell mortality assays. The cell lysates were immunoblotted using the APP antibody, 22C11.

FIG. 23 shows CLSPCOL efficiently passes the blood-brain barrier. (a) The absorbance of stepwise increasing concentrations of wt-CLSPCOL and CLSPCOL was measured at 450 nM to simulate standard dose-response lines as shown in Table L1. (b) At an hour after intraperitoneal injection of 10 nmol GST-MycHisG, CLSPCOL, and wt-CLSPCOL, brains and serum were harvested from mice for ELISA. As shown in Table L1, the concentrations of CLSPCOL and wt-CLSPCOL in interstitial fluid (ISF)-containing brain lysates and serum were measured using ELISA. (c) The ratio of ISF to serum concentrations was calculated and indicated.

FIG. 24 shows CLSPCOL is not inhibited by ApoE3 and 14-3-3σ but is slightly inhibited by calreticulin. SH-SYSY cells were transfected with the pcDNA3.1/MycHis vector (vector) or pcDNA3.1/MycHis-V642I-APP (V642I-APP). The cells were then cultured in DMEM/F12-10% FBS containing 100 pM GST-MycHis or CLSPCOL and 1 nM ApoE3, 14-3-3σ, or calreticulin. At 24 hours after the transfection, the media were replaced with DMEM/F12 with N2 supplement containing the same concentration of reagents. At 48 hours after the onset of the transfection, the cells were harvested to perform the trypan blue exclusion cell mortality assays. The cell lysates were immunoblotted using the APP antibody, 22C11.

FIG. 25 shows CLSPCOL begins to be inhibited by a 10-fold or more higher concentration of calreticulin. SH-SYSY cells were transfected with the pcDNA3.1MycHis vector (vector) or pcDNA3.1/MycHis-V642I-APP (V642I-APP). The cells were then cultured in DMEM/F12-10% FBS containing GST-MycHis (1 nM), CLSP1-61-MycHis (1 nM), CLSPCOL (100 pM), or wt-CLSPCOL (100 pM) and indicated concentrations of calreticulin or BSA. At 24 hours after the transfection, the media were replaced with DMEM/F12 with N2 supplement containing the same concentration of reagents. At 48 hours after the onset of the transfection, the cells were harvested to perform the trypan blue exclusion cell mortality assays. The cell lysates were immunoblotted using the APP antibody, 22C11.

### DESCRIPTION OF EMBODIMENT

[CLSP derivative]

An amino acid sequence (I) consisting of 22 amino acids (amino acid 40-61) included in calmodulin-like skin protein (CLSP) (amino acid sequence 1): TGKNLSEAQLRKLISEVDS(or G)DGD (amino acid single letter code) (I) is called Endogenous Humanin-Homogenous Region (EHR) or Endogenous Humanin-Like Domain (EHD), and plays a core role in the CLSP-mediated suppression of neuronal cell death (Patent Literature 1).

The CLSP derivative of the present invention is characterized by including the endogenous humanin-homogenous region (EHR), which is the core of an activity to suppress Alzheimer's disease-related neuronal cell dysfunction or neuronal cell death (CLSP activity or CLSP suppressive activity), and not including a region to which an inhibitor or inhibiting substance of the activity (CLSP inhibitor) binds. Examples of the region to which the inhibitors bind can include the C-terminal amino acid sequence region (amino acid 62-146) of CLSP (SEQ ID NO: 1).

The “activity to suppress Alzheimer's disease-related neuronal cell dysfunction or cell death” in the present invention indicates the suppression or antagonization of at least one of neuronal cell dysfunction or cell death regardless of their causes or causal relationships. The suppression of neuronal cell death may be not full suppression but rather significant suppression. The neuronal cell death-suppressing activity can be evaluated in accordance with methods described in Examples below or methods described in other documents (e.g., see International Patent Number WO00/14204). The CLSP activity can be measured, for example, as the V642I-APP-induced neuronal cell death-suppressing activity using various neuronal cell death assays.

Furthermore, the binding between an inhibitor and CLSP can be measured using any method and means (assay system) known by those skilled in the art as described in Examples of the description. It can be measured, for example, by immunoblot analysis, pull-down analysis, Nano-Glo HiBiT Extracellular Detection System, and ELISA and the like.

Specific examples of EHR can include the amino acid sequence (I): TGKNLSEAQLRKLISEVDS(or “G”)DGD (amino acid single letter code) (I), or an amino acid sequence consisting of 22 amino acids described in claim 1 of Patent Literature 1. Furthermore, examples of the region to which the inhibitors bind can include the C-terminal amino acid sequence region (amino acid 62-146) of CLSP (SEQ ID NO:1).

Therefore, suitable examples of the CLSP derivative of the present invention can include a polypeptide consisting of an amino acid sequence below:

(1) an N-terminal amino acid sequence region (amino acid 1-61) of CLSP;

(2) an amino acid sequence of (derived from) the (1) above, wherein one or several (e.g., about 2-5) amino acids are deleted, substituted or inserted in an amino acid sequence other than EHR included in the amino acid sequence of the (1) above; or

(3) an amino acid sequence of (derived from) the (1) above, which has an identity of 90% or more, preferably 95% or more, and further preferably 98% or more to an amino acid sequence other than EHR included in the amino acid sequence of the (1) above.

The CLSP derivative of the present invention is characterized by having the CLSP activity equivalent to that of wild-type CLSP and being substantially (significantly) insensitive to the inhibitory or suppressive action by the inhibitors of the CLSP activity because the derivative does not include the region to which the inhibitors bind. It should be noted that the CLSP derivative of the present invention includes, for example, fusion proteins (hybrid polypeptides) including various mutants such as deletion mutants and EHR, and the like, but does not include a polypeptide consisting of only EHR.

The CLSP inhibitors are not particularly restricted to their structural features, etc. They are, for example, substances to show a significant inhibitory (suppressive) effect on the CLSP activity at a concentration equivalent to or 5-fold or more the concentration of CLSP in media, and are, for example, selected from the group consisting of apolipoprotein E (ApoE), 14-3-3 proteins, and calreticulin. Particularly, the CLSP activity-suppressing effect of ApoE (ApoE3 and ApoE4) was shown to be high.

### [Adiponectin and its Derivative]

Furthermore, the present invention revealed that adiponectin (SEQ ID NO:3) had CLSP activity-potentiating action/effect by binding to EHR in the CLSP1-61 region of CLSP and the CLSP derivative of the present invention by a polypeptide (SEQ ID NO:2), which is the collagen-homologous region of adiponectin (ADNCol), and moreover that adiponectin and the polypeptide also had an action to protect the CLSP from the inhibitory or suppressive action by the above inhibitors of the CLSP activity or nullify the action by the inhibitors.

Therefore, the polypeptides (hereinafter also referred to as “adiponectin and its derivative”) consisting of an amino acid sequence below:

(1) an amino acid sequence (ADNCol) shown by SEQ ID NO:2;

(2) an amino acid sequence including the amino acid sequence (ADNCol) of the (1) above, for example, adiponectin shown by SEQ ID NO:3;

(3) an amino acid sequence of (derived from) adiponectin shown by SEQ ID NO:3, wherein one or several (e.g., about 2-5) amino acids are deleted, substituted or inserted in an amino acid sequence other than ADNCol included in the amino acid sequence shown by SEQ ID NO:3; or

(4) an amino acid sequence of (derived from) adiponectin shown by SEQ ID NO:3, which has an identity of 90% or more, preferably 95% or more, and further preferably 98% or more to an amino acid sequence other than ADNCol included in the amino acid sequence shown by SEQ ID NO:3;

are useful as a potentiator or protector of the CLSP activity by CLSP or the CLSP derivative of the present invention. It should be noted that these polypeptides may be, for example, a multimer like trimeric adiponectin.

It should be noted that “CLSP” also contains various polypeptides related (similar) to CLSP which have the CLSP activity as described in Patent Literature 1 in addition to the polypeptide shown by SEQ ID NO:1 described in the description. In addition, “suppression” and “inhibition” on Alzheimer's disease-related neuronal cell dysfunction or neuronal cell death have the same meaning about such CLSP activity. Furthermore, “protect,” “keep” and “retain” have the same meaning about the activity of the potentiator or protector of the present invention.

### [Fusion Protein of e.g., the CLSP Derivative and e.g., the Adiponectin Derivative]

The present invention further relates to a fusion protein (hybrid polypeptide) including CLSP or the CLSP derivative, an example of the above CLSP derivative, and adiponectin or the adiponectin derivative. The fusion protein has potent CLSP activity and is insensitive to the suppression by CLSP inhibitors or, even when it is suppressed, the degree is moderate.

Particularly a fusion protein (CLSPCOL) consisting of the N-terminal amino acid sequence region (amino acid 1-61) of CLSP and ADNCol, a suitable example, can penetrate efficiently the blood-brain barrier and be transferred to the CNS.

The involved fusion protein can optionally include an amino acid sequence other than polypeptides forming the above regions (elements) as long as the predetermined activity of the fusion protein is not destroyed. A linker sequence consisting of an appropriate amino acid sequence can be inserted between regions, for example, for the purpose of improving the stability of the three-dimensional structure of protein and the like. Alternatively, an optional amino acid sequence known by those skilled in the art, such as constant regions of immunoglobulin found in known fusion proteins, can be also added to the C-terminal side, for example, for the purpose of improving in vivo stability and the like (e.g., half-life in the blood plasma).

Those skilled in the art can appropriately design and prepare such added or inserted sequences based on common technical knowledge while considering e.g., antigenicity. Because CLSP1-61 and the collagen-homologous region are derived from human endogenous peptides, their antigenicity is presumed to be limited.

Furthermore, the order (N-terminal side or C-terminal side) of connecting regions included in fusion proteins is not particularly restricted and can be appropriately selected and prepared by those skilled in the art.

The CLSP derivative, adiponectin and its derivative, potentiator or protector of the CLSP activity by CLSP or the CLSP derivative, which the potentiator or protector consists of a polypeptide, and polypeptide forming a fusion protein of the present invention will be simply referred to as “the polypeptide of the present invention.”

About the polypeptide of the present invention, the sequences are pretreated to suitable states for comparison to determine sequence identity of two amino acid sequences. For example, by inserting a gap in one sequence, the alignment with the other sequence is optimized. Amino acid residues or bases in each site are then compared. When amino acid residues or bases in a site in the first sequence exist in the corresponding site of the second sequence, these sequences are identical in the site. The identity of two sequences is shown as the percentage of the number of identical sites between sequences to the number of all sites (all amino acids or all bases).

In accordance with the above principle, the identity of two amino acid sequences can be determined by any method known by those skilled in the art. The identity can be determined, for example, by the algorithms of Karlin and Altshul (Proc. Natl. Acad. Sci. USA 87: 2264-2268, 1990 and Proc. Natl. Acad. Sci. USA 90: 5873-5877, 1993). BLAST program using such algorithms was developed by Altshul et al. (J. Mol. Biol. 215: 403-410, 1990).

Furthermore, Gapped BLAST is a program which determines identity with better sensitivity than BLAST (Nucleic Acids Res. 25: 3389-3402, 1997). The above programs are mainly used to search sequences showing high identity to a given sequence from the database. These can be used, for example, on the Internet website of National Center for Biotechnology Information in USA.

Alternatively, a value determined using BLAST 2 Sequences software developed by Tatiana A. Tatusova and others (FEMS Microbiol Lett., 174: 247-250, 1999) can be also used as identity between sequences. This software can be used and available on the Internet website of National Center for Biotechnology Information in USA. The programs and parameters used are as follows. In the case of amino acid sequences, blastp program is used and the parameters were Open gap: 11 and extension gap: 1 penalties, gap x_dropoff: 50, expect: 10, word size: 3, Filter: ON. Furthermore, a sequence showing identity can be also searched from database using a high-sensitive FASTA software (W. R. Pearson and D. J. Lipman, Proc. Natl. Acad. Sci. USA, 85: 2444-2448, 1998). All parameters are used as default values on the website.

The polypeptide of the present invention described above can also have an altered form by e.g., modification, addition, mutation, substitution or deletion by known methods. Such alteration of functional groups can be performed using any method known by those skilled in the art, for example, for the purpose of protecting the polypeptide, controlling the stability or tissue penetration of the polypeptide, or controlling the activity of the polypeptide, or the like.

That is, the polypeptide of the present invention may be naturally modified by e.g., posttranslational modification. They may be also artificially modified. Modifications include those of the peptide backbone, amino acid side chains, the amino terminal or the carboxyl terminal and the like. In addition, the polypeptide may be branched or cyclic. Modifications include, but not limited to, acetylation, acylation, ADP-ribosylation, amidation, covalent binding of e.g. [flavin, nucleotide, a nucleotide derivative, lipid, a lipid derivative, or phosphatidylinositol], cross-link formation, circularization, di sulfide bond formation, demethylation, pyroglutamylation, carboxylation, glycosylation, hydroxylation, iodination, methylation, myristoylation, oxidation, phosphorylation, ubiquitination and the like. Furthermore, the above peptides or polypeptides can be in the form of any salt or ester known by those skilled in the art.

Furthermore, the polypeptide of the present invention can also form a fusion polypeptide with any known neurotropic peptide, and such fusion polypeptide can be easily synthesized by any method known by those skilled in the art.

The polypeptide of the present invention can be prepared from e.g., cell lines derived from appropriate species, such as human and mice, based on gene or amino acid sequence information related to e.g., CLSP and adiponectin known by those skilled in the art, and moreover can be produced by a known technique for synthesizing a peptide. In addition, the polypeptide of the present invention can be produced by introducing and expressing e.g., a vector including DNA encoding these into e.g., appropriate host cells using a genetic engineering technique known by those skilled in the art. At this time, for example, in the case of a CLSP derivative or an adiponectin derivative, they are prepared by properly altering a part of their amino acid sequences by a method/means known by those skilled in the art.

Such vector has any form known by those skilled in the art, such as a plasmid or virus vector, and can be easily prepared by any method known by those skilled in the art. The vectors thus obtained properly include, in addition to the coding region of site-specific recombinant enzymes of the present invention, non-coding sequences (including e.g., a nuclear transport signal, tag sequence, non-transcribed sequence, untranslated sequence, promoter, enhancer, suppressor, transcriptional factor-binding sequence, splicing sequence, poly A-additional sequence, IRES, mRNA stabilizing/destabilizing sequence) at 5′ and 3′, and function as an expression vector.

Appropriate host cells can be easily transformed by any method known by those skilled in the art using such vector, for example, a lipofection method, a calcium phosphate method, and various physical methods such as electroporation and particle gun.

The host cells are not particularly restricted, and, for example, mammalian cells including human, monkeys and mice, plant cells, insect cells and bacteria such as E. coli can be used. The transformed cells thus produced are cultured on optional conditions known by those skilled in the art, and e.g., the polypeptide of the present invention of interest can be easily prepared from an appropriate fraction such as cultured bacterial cells or its cultured supernatant.

The polypeptide of the present invention is useful as an active ingredient of a pharmaceutical composition to suppress Alzheimer's disease-related neuronal cell dysfunction or neuronal cell death, for example, a pharmaceutical composition used to prevent or treat diseases accompanied by Alzheimer's disease-related memory impairment or neurodegeneration.

Furthermore, the polypeptide of the present invention can be used to prevent and treat, in addition to Alzheimer's disease, diseases accompanied by memory impairment or neurodegeneration, for example, disorders caused by neuronal cell death due to cerebral ischemia (T. Kirino, 1982, Brain Res., 239: 57-69). Besides, Parkinson's disease accompanied by dementia (M. H. Polymeropoulos et al., 1997, Science, 276: 2045-2047), diffuse Lewy bodies (M. G. Spillantini et al., 1998, Proc. Natl. Acad. Sci. USA, 95: 6469-6473), dementia accompanied by Down syndrome, and the like are also treated and prevented. In addition, because APLP1, a related molecule of APP, is said to be the causal gene of congenital nephrotic syndrome (Lenkkeri, U. et al., 1998, Hum. Genet. 102: 192-196), kidney disorders such as nephrotic syndrome are also treated and prevented.

The pharmaceutical composition of the present invention can be also formulated by a known pharmaceutical method in addition to directly administering the active ingredient itself to patients. The pharmaceutical composition of the present invention is considered to be formulated by proper combination with, for example, a pharmacologically acceptable carrier or medium, specifically sterile water, saline, plant oil, an emulsifier, a suspension agent, a surfactant, a stabilizer, a slow-release agent and the like, and administered. The pharmaceutical composition of the present invention can be in the form of e.g., aqueous solutions, tablets, capsules, troches, buccal tablets, elixirs, suspension, syrups, nasal solutions, or inhalant liquid. The percentage of a peptide or polypeptide, the active ingredient, included may be properly determined depending on e.g., intended uses and formulation forms.

The pharmaceutical composition of the present invention can be administered to patients, for example, but not limited to, transdermally, intranasally, transbronchially, intramuscularly, intraperitoneally, intravenously, intrathecally, intraventricularly, or orally depending on the properties of active ingredient. When used to treat brain neurodegenerative disorders, the pharmaceutical composition of the present invention is desirably introduced to the central nervous system via any appropriate route including intravenous, intrathecal, intraventricular or intradural injection. The dosage and administration method vary depending on the tissue penetration of the active ingredient in the pharmaceutical composition of the present invention, therapeutic purposes, the weight, age and symptom of patients, and the like, but can be properly selected by those skilled in the art. For example, medicine can be administered at several tens of μ1 per one treatment once to several times daily over an appropriate period of time. The active ingredient can have a concentration, for example, from about 10 pmol to 100 nmol.

As described above, the pharmaceutical composition of the present invention can be widely used to prevent or treat disorders accompanied by neuronal cell dysfunction or neuronal cell death, such as Alzheimer's disease, or diseases accompanied by memory impairment or neurodegeneration.

Therefore, the present invention relates to a method for suppressing neuronal cell dysfunction or cell death, the method including the step of bringing the polypeptide of the present invention into contact with neuronal cells, and a method for treating a disorder accompanied by neuronal cell dysfunction or neuronal cell death, such as Alzheimer's disease, or a disease accompanied by memory impairment or neurodegeneration, the method including the stage of administering the pharmaceutical composition of the present invention to a subject (individual), an animal such as human, affected with or suspected of the disorder or the disease, and a method for treating disorders accompanied by neurodegenerative disorders.

The present invention further relates to a method for detecting an activity to suppress neuronal cell dysfunction or neuronal cell death by the polypeptide of the present invention, the method including (a) the step of inducing the neuronal cell dysfunction or cell death in the presence/absence of a CLSP inhibitor, and in the presence/absence of the polypeptide, (b) the step of detecting the neuronal cell dysfunction or cell death, and (c) the step of comparing the neuronal cell dysfunction or neuronal cell death in the presence/absence of the polypeptide, and the like.

By the above method, the activity to suppress the neuronal cell dysfunction or cell death and the activity to potentiate or protect the CLSP activity by CLSP or the CLSP derivative from CLSP inhibitors can be detected by the polypeptide of the present invention.

Specific operations can be performed, for example, in accordance with a method described in the description. This method can be used to determine whether or not the polypeptide of the present invention have the suppressive effect on cell death in various cells, and quantify the suppressive effect. The cells are not particularly restricted and various cells which can cause cell death are used. In addition, for the induction of cell death, known cell death-inducing systems can be used depending on respective cells. In addition, the method can be used to detect the effect of e.g., the polypeptide of the present invention in various conditions such as various stimulations, changes in environment or gene expression to induce neuronal cell death, using neuronal cells. In addition, such detection can be used to detect differences in sensitivity to e.g., the polypeptide of the present invention in neuronal cell death, which can exist between species, subspecies or individuals. Because of this, the effectiveness of the polypeptide of the present invention, for example, between ethnic groups, races, or individuals can be examined. By such method, for example, detailed conditions toward clinical applications can be examined.

The present invention also relates to a method for screening a substance (test substance) regulating an activity to suppress neuronal cell dysfunction or neuronal cell death by the polypeptide of the present invention or CLSP. This method can be used to assay the effect (influence) of the test substance on the activity to suppress the neuronal cell dysfunction or neuronal cell death by the polypeptide of the present invention or CLSP. The polypeptide of the present invention or CLSP is considered to act the surface of neuronal cells to exert the cell death-suppressing effect. Using this method, the action of candidate compounds which can inhibit or conversely promote the contact of these polypeptides on the surface of cells can be inspected.

This screening method includes (a) the step of inducing the neuronal cell dysfunction or neuronal cell death with or without a test substance in the presence of the polypeptide of the invention or CLSP, (b) the step of detecting the neuronal cell dysfunction or neuronal cell death, and (c) the step of selecting a substance regulating the activity to suppress the neuronal cell dysfunction or neuronal cell death by the polypeptide of the invention or CLSP. In the step (c), comparison with any control can be performed. In the step (c), for example, compounds that promote or suppress the neuronal cell dysfunction or neuronal cell death in the presence of a test substance can be selected by comparison with the case in the absence of the test substance. Compounds that promote the neuronal cell dysfunction or neuronal cell death become candidate compounds that inhibit the action by the polypeptide of the present invention or CLSP, and compounds that further suppress the neuronal cell death become candidate compounds that further promote the action by the polypeptide of the present invention or CLSP.

In the above screening, a compound different from the test substance can be also used as a control. For example, in the step (b), another compound which can regulate the suppression of neuronal cell dysfunction or neuronal cell death by the polypeptide of the present invention or CLSP is used for detection, and in the step (c), compared with a case in the presence of the compound, compounds that further promote or suppress the neuronal cell dysfunction or neuronal cell death in the presence of the test substance used in the step (a) can be selected. In such screening, compounds that have even higher ability to regulate the suppression of the neuronal cell dysfunction or neuronal cell death by the polypeptide of the present invention or CLSP than of existing compounds can be screened.

Examples of the test substance used in the above screening include, but not limited to, purified proteins (including antibodies), gene library expression products, synthetic peptide libraries, cell extract, cell culture supernatant, synthetic low molecular weight compound libraries, natural materials such as soil, and solutions including a bacterium-releasing substance such as actinomycete broth, and the like. Induction of the neuronal cell death and administration of the polypeptide of the present invention can be performed in accordance with any method known by those skilled in the art.

The time to apply a test substance to cells is not particularly restricted, and it can be applied before, after or simultaneously with the polypeptide of the present invention. The method for applying a test sample is not restricted, and, for example, it is added to media in the case of cultured cell systems. In addition, in the case of nucleic acids, it may be introduced into cells. A test sample can be applied by other optional administration methods.

Substances evaluated by the above examination on the actions of compounds or substances obtained by screening become candidate compounds that regulate the activity of the polypeptide of the present invention, and are considered to be applied to prevent and treat disorders including Alzheimer's disease.

The present invention further relates to a method for screening a substance (compound) which binds to the polypeptide of the present invention, the method including (a) the step of bringing a test substance into contact with the polypeptide, (b) the step of detecting binding activity of e.g., the polypeptide and the test substance, and (c) the step of selecting a substance having the activity to bind to the polypeptide.

The polypeptide of the present invention can be used for screening as a soluble polypeptide or a form binding to a carrier depending on screening techniques. The polypeptide of the present invention may be labeled. Examples of labeling include radioactive isotope labeling, fluorescent substance labeling, biotin or digoxigenin labeling, tag sequence addition, and the like.

Examples of the test substance used for screening include, but not limited to, purified proteins (including antibodies), gene library expression products, synthetic peptide libraries, cell extract, cell culture supernatant, synthetic low molecular weight compound libraries, natural materials such as soil, and solutions including a bacterium-releasing substance such as actinomycete broth, and the like. The test substance is used after proper labeling as needed. Examples of labeling include, but not limited to, radiolabeling, fluorescent labeling and the like.

For example, when screening a protein which binds to the polypeptide of the present invention, the protein binding to the polypeptide of the present invention can be screened by applying a tissue or cell extract, which is expected to have expressed the protein which binds to the polypeptide of the present invention, to an affinity column to which the polypeptide of the present invention is fixed, and purifying the protein which specifically binds to the column.

A cDNA library using a phase vector is further created from a tissue or cells (e.g., cerebral cortex tissue or neuronal cells such as F11) which are expected to have expressed a protein which binds to the polypeptide of the present invention, plaques are formed on agarose, and screening can be performed by the western blotting method using e.g. the polypeptide of the present invention which is labeled. Also, screening can be performed in accordance with e.g. the “two hybrid system” in which a DNA-binding peptide such as the GAL4 DNA-binding region, and a transcription-activating peptide such as the GAL4 transcription-activating region are expressed as fusion proteins with e.g., the polypeptide of the present invention and a test protein respectively to detect the binding between e.g., the polypeptide of the present invention and the test protein through the expression of a reporter gene linked to the downstream of a promoter having the binding sequence of the DNA-binding peptide.

Cloning a receptor to the polypeptide of the present invention is also considered by the screening of the present invention. In this case, a test sample is preferably prepared from a tissue or cells which are expected to have expressed the receptor, such as cerebral cortex tissue, a neuronal cell line, or neuroblastoma and teratoma cells, and the like. Examples of neuronal cell lines include F11 cells, PC12 cells (L. A. Green and A. S. Tischler, 1976, Proc. Natl. Acad. Sci. USA, 73: 2424-2428), NTERA2 cells (J. Skowronski and M. F. Singer, 1985, Proc. Natl. Acad. Sci. USA, 82: 6050-6054), SH-SY5Y cells (L. Odelstad et al., 1981, Brain Res., 224: 69-82), and the like.

A synthetic compound, natural product library, or random phage peptide display library or the like may be allowed to act on the polypeptide of the present invention which has been fixed, to screen a molecule which binds to the peptide. Screening by detecting bindings using the surface plasmon resonance phenomenon is also capable (e.g. Biacore (manufactured by BlAcore) and the like). These screening can be also performed by high throughput screening using a combinatorial chemistry technology.

A compound which binds to the polypeptide of the present invention, obtained by the screening of the present invention becomes a candidate compound that regulates the activity of the polypeptide of the present invention, and is considered to be applied to prevent and treat disorders including Alzheimer's disease.

The present invention will now be described in further detail by way of Examples thereof. It should be noted that the technical scope of the present invention is not restricted in any way by these descriptions.

### EXAMPLE

### [Identification of Multiple CLSP Interactors]

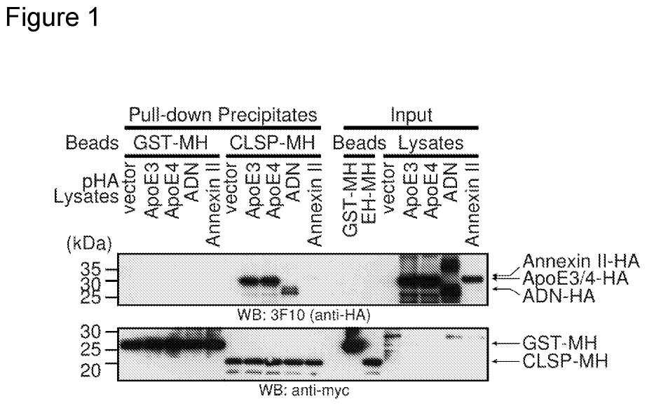

Multiple proteins such as 14-3-3σ, 14-3-3β, calreticulin, ERp27, nucleolin, annexin II, and annexin V were identified as putative CLSP binding factors in a previous study (15). Among these, proteins which have been reported to be secreted into the extracellular space were selected for analyses in the present invention. In addition, the present invention newly found that apolipoprotein E (ApoE) and adiponectin bound to CLSP (FIG. 1).

### [Apolipoprotein E, 14-3-3 Proteins and Calreticulin are CLSP Inhibiting Substances]

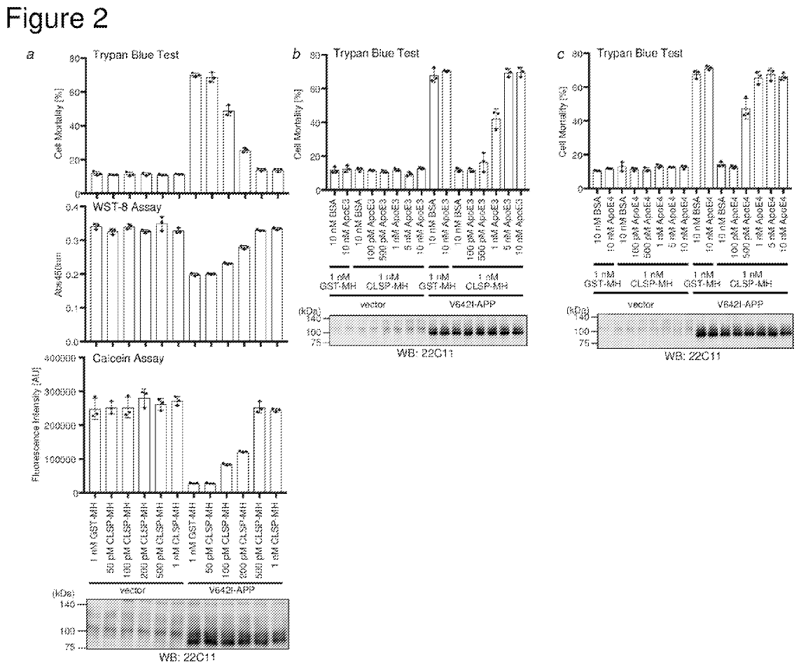

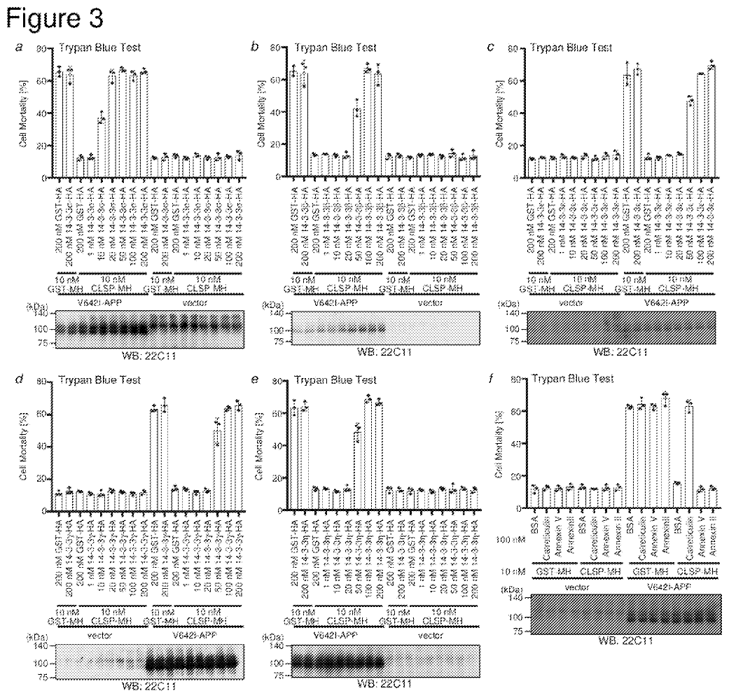

As previously described (5), overexpression of V642I-amyloid _{R} precursor protein (V642I-APP) caused SH-SYSY neuroblastoma cell death but the co-incubation with recombinant CLSP (500 pM or 1 nM) produced in bacteria completely suppressed the V642I-APP-induced neuronal death (FIG. 2a). The dose-response analysis showed that the 50% effective concentration of CLSP produced in bacteria was estimated to be about 200 pM (FIG. 2a) and was slightly larger than of recombinant CLSP produced in mammal cells (5). Interestingly, the addition of recombinant apolipoprotein E3 or E4 (ApoE3 or ApoE4) to media inhibited the CLSP-mediated protection of the V642I-APP-induced neuronal death in a dose-response manner (FIGS. 2b and c). ApoE3 completely inhibited the effect of 1 nM CLSP at a concentration of 5 nM (FIG. 2b) whereas ApoE4 completely inhibited the effect of 1 nM CLSP at a concentration of 1 nM (FIG. 2c). This result indicates that the inhibitory effect of ApoE4 is slightly stronger than of ApoE3. Similarly, the co-incubation with recombinant 14-3-3σ inhibited the CLSP-mediated protection of V642I-APP-induced neuronal death (FIG. 3a). The inhibitory effect by 14-3-3σ against 10 nM CLSP began to be observed at a concentration of 10 nM and complete inhibition was obtained by the addition of 20 nM recombinant 14-3-3σ in this specific experiment. Other 14-3-3 proteins also inhibited the CLSP-mediated inhibition of V642I-APP-induced neuronal cell death and complete inhibition was obtained by 50 nM recombinant 14-3-3σ (FIGS. 3b-e). Similarly, 50 nM calreticulin completely inhibited the effect of 10 nM CLSP (FIG. 3f). Therefore, these results demonstrate that CLSP inhibitors show full CLSP-inhibiting effect at a concentration almost equal to or 5-fold or more higher than the concentration of CLSP in media. In contrast, annexin II, annexin IV or adiponectin did not show any inhibitory activity even at a concentration 5-fold or 10-fold higher than the concentration of CLSP in media (FIG. 3f and Fig. S-1).

### [Adiponectin Keeps CLSP Active in the Presence of Much Higher Concentrations of CLSP Inhibiting Substances]

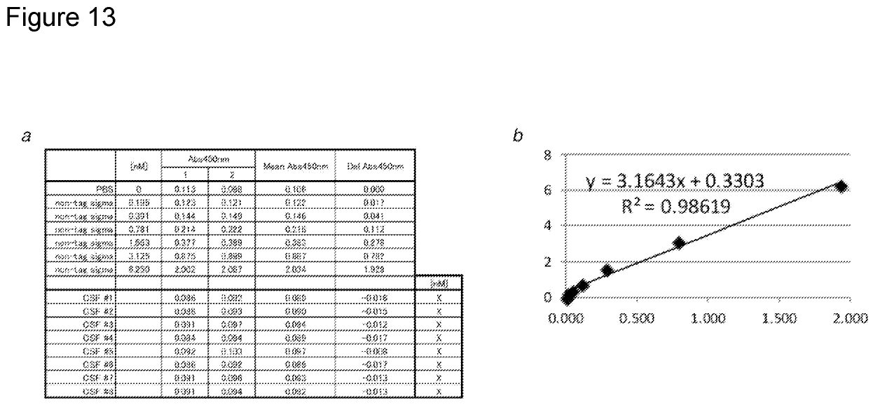

The concentration of CLSP in human cerebrospinal fluids (CSF) is estimated to be 3-6 nM (14). It is known that ApoE is produced from astrocytes and microglia and a large proportion of ApoE is recruited to form a high-density lipoprotein-like lipoprotein simultaneously with lipid and other apolipoproteins in human CNS (16, 17). The concentration of ApoE in the human CSF is estimated to be 40 to 200 nM (18-20). On the other hand, the concentration of 14-3-3σ in the human CSF was estimated to be much lower than 1 nM (see FIG. 13). The concentration of 14-3-3γ in the human CSF is estimated to be lower than 1 nM by a previous study (21). Furthermore, the concentration of calreticulin in human serum was estimated to be nearly 10 pM (22), whereas the concentration in the human CSF has not been measured until now. In summary, the concentration of ApoE as a total amount is 10-fold or more higher than the concentration required to inhibit the CLSP function completely, whereas the concentrations of other inhibiting substances are likely insufficient to inhibit CLSP in the human CNS.

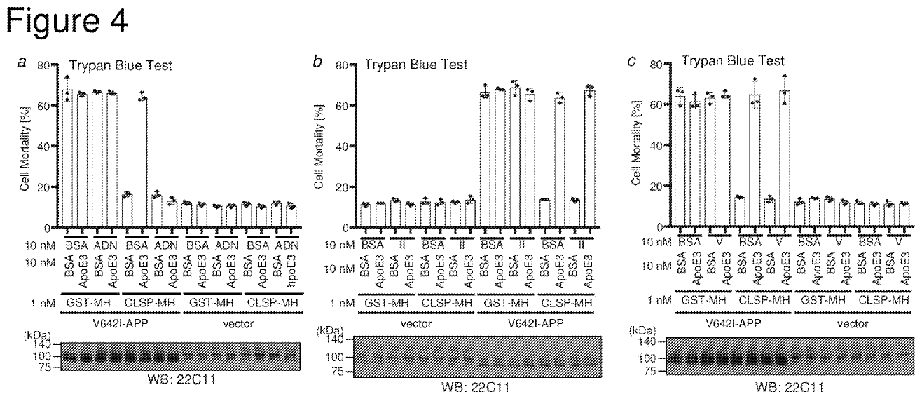

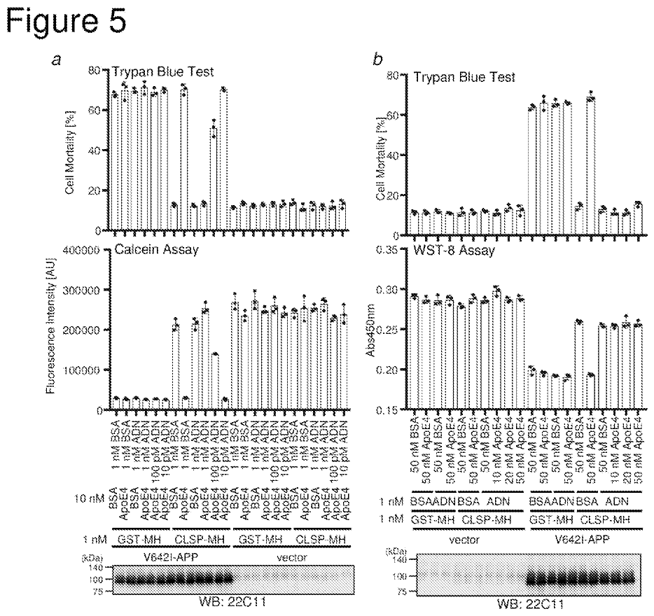

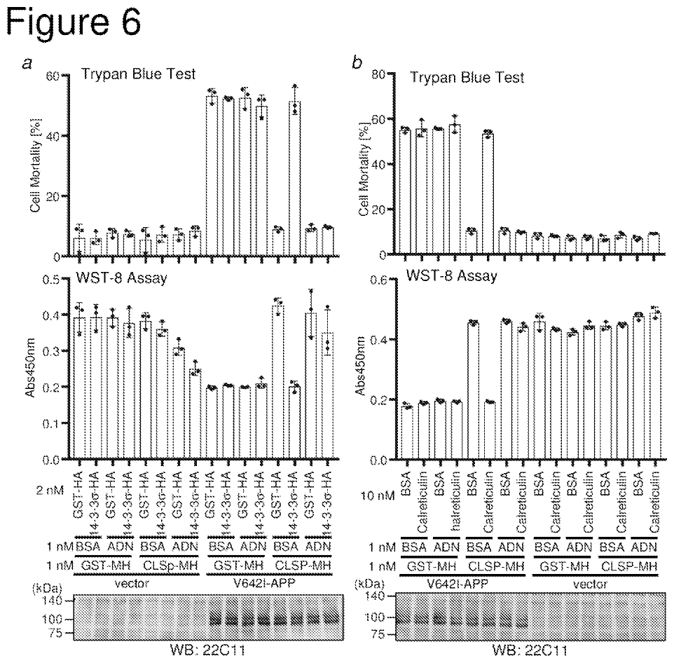

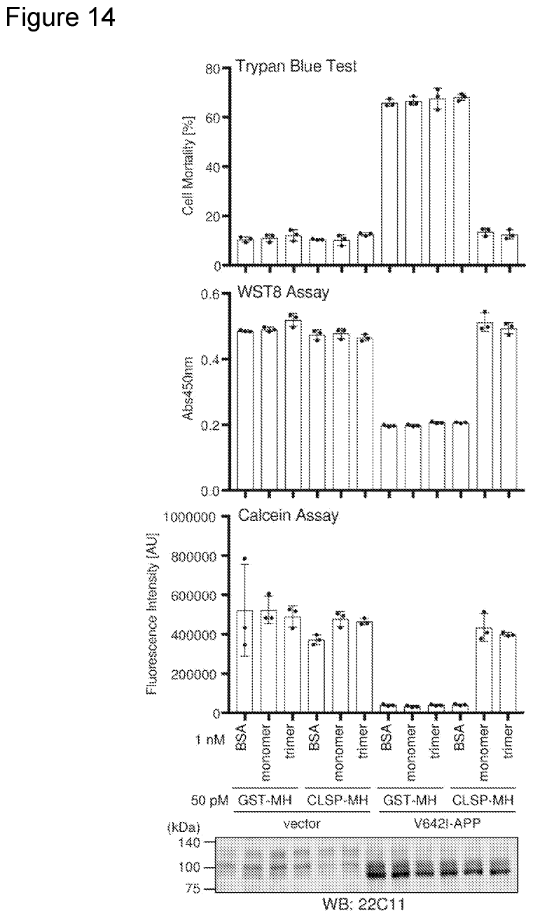

As described above, because a very high amount of CLSP inhibiting substance (mainly consisting of ApoE) exists in the human CNS, the CLSP activity appears zero in vivo. It is, however, natural to think that the CLSP activity exists at least in healthy people according to a previous examination (35). Therefore, we next examined a hypothesis that any of other CLSP-binding substances protected CLSP from CLSP-inhibiting substances to keep the activity. Because of this, recombinant proteins of annexin II, annexin V or adiponectin, candidates, were added to cell death assay systems including CLSP (1 nM) and ApoE3 (10 nM) at a concentration equal to that of ApoE3. As a result, adiponectin completely nullified the ApoE3-mediated inhibition of the CLSP activity (CLSP-protecting activity) (FIG. 4a). Neither annexin II nor annexin V showed such neutralization activity at the tested concentrations (FIGS. 4b and c). The concentration of adiponectin was then reduced stepwise to determine the minimal concentration of adiponectin that nullifies the inhibition. As a result, adiponectin completely suppressed the CLSP-inhibiting activity of ApoE4 at the following concentration ratio (CLSP, 1 nM; ApoE4, 10 nM: adiponectin, 1 nM) (FIG. 5a). Furthermore, adiponectin partially suppressed the CLSP-suppressing activity of ApoE4 even at a concentration of 100 pM, which is further reduced (CLSP, 1 nM: ApoE4, 10 nM: adiponectin, 100 pM). In contrast, even an increase in the concentration of ApoE4 up to 50 nM did not attenuate the protective effect of 1 nM adiponectin at all (FIG. 5b). These results indicate that 1 nM adiponectin keeps the concentration of active CLSP at the 100% effective level even in the presence of a much higher concentration of ApoE4. Wild-type adiponectin is multimerized spontaneously in vivo to form 3 types of multimers. Trimer, hexamer and octamer or more are referred to as low-molecular-weight adiponectin, middle-molecular-weight adiponectin, and high-molecular-weight adiponectin, respectively (23). From a previous study it is known that middle or high-molecular-weight adiponectin usually plays a core role in metabolic activity via adiponectin receptors (23). The study found that, using a recombinant trimeric adiponectin that does not form middle-molecular-weight or high-molecular-weight adiponectin, the trimeric adiponectin had CLSP-potentiating effect similar to that of wild-type adiponectin (FIG. 14). This result means that the multimerization of adiponectin to middle-molecular-weight or high-molecular-weight is not essential for the CLSP-protecting effect of adiponectin. From the previous study it is known that adiponectin multimerized to middle-molecular-weight or high-molecular-weight shows higher activity about metabolic functions via canonical adiponectin receptors (23). In summary, the obtained findings strongly support a possibility that the CLSP-protecting activity of adiponectin found herein is not an effect via canonical adiponectin receptors. The similar examinations about CLSP inhibiting substances other than ApoE further found that adiponectin completely showed the protective activity on the CLSP-inhibiting effect by 14-3-3σ or calreticulin (CLSP, 1 nM: 14-3-3σ or calreticulin, 2 nM or 10 nM: adiponectin, 1 nM) (FIG. 6).

### [Adiponectin Potentiates the CLSP Activity]

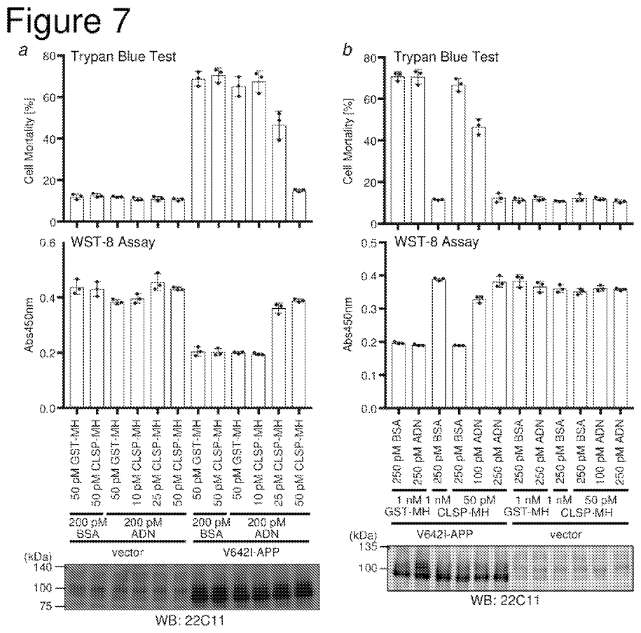

It was further found that adiponectin also had the effect of potentiating CLSP activity itself in addition to the above CLSP-protecting effect. As shown in FIG. 2a, CLSP did not show V642I-APP-induced cell death-suppressing activity at a concentration of 50 pM. However, CLSP showed nearly full or partial cell death-suppressing activity at a concentration of 50 pM or 25 pM respectively in the presence of 200 pM adiponectin (FIG. 7a). This result indicates that adiponectin binds to CLSP to potentiate the CLSP activity. The present inventors further found that even when the concentration of adiponectin concurrently administered was reduced to 100 pM, adiponectin showed partially potentiating activity on CLSP at the concentration of 50 pM (FIG. 7b). In summary, these results indicate that the minimal concentration of adiponectin that provides full cell death-suppressing activity to 50 pM CLSP, which alone does not have the activity, is 200 to 250 pM.

### [The Dissociation Constant for the Binding Between Adiponectin and CLSP is Near the Dissociation Constant for the Binding Between Apolipoprotein E4 and CLSP]

As described above, it was described that adiponectin completely nullified the CLSP activity-inhibiting effect of a 50-fold higher concentration of ApoE (protective effect) (FIG. 4 and FIG. 5). Such potent CLSP-protecting effect imparted to adiponectin can be explained by two mechanisms described below. First, adiponectin competes with ApoE for binding to the same site of CLSP and the binding affinity between adiponectin and CLSP is much stronger than that between apolipoprotein E and CLSP (competitive antagonist). Second, adiponectin binds to a region of CLSP that is different from the region to which ApoE binds, to elevate the CLSP activity in the case of single binding, and when there is binding of a CLSP inhibitor simultaneously, to suppress the inhibitory effect and keep the CLSP activity (noncompetitive antagonist).

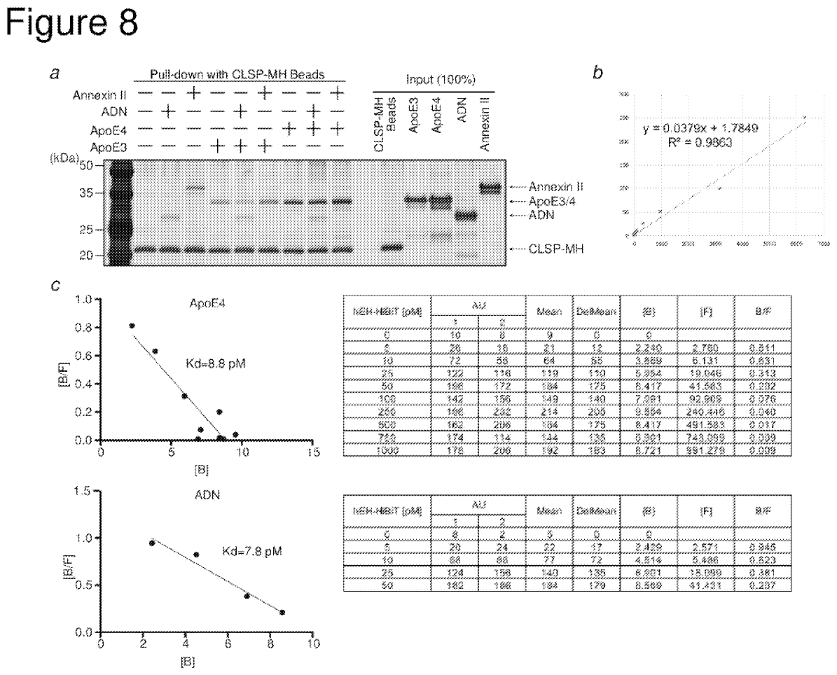

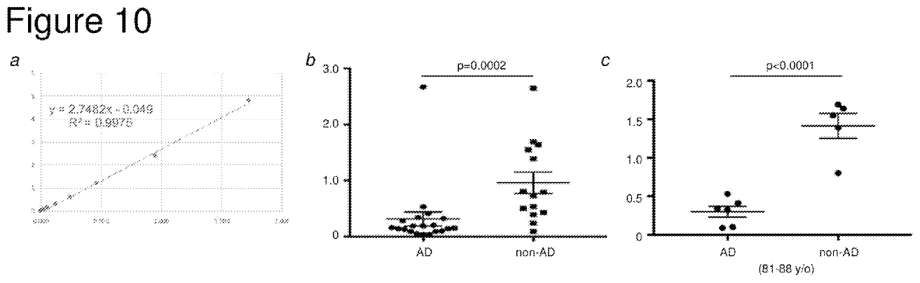

It was examined which of these two mechanisms is correct. First, in order to observe how the presence of ApoE3 or ApoE4 affects CLSP and adiponectin or annexin II and vice versa, sepharose 4B beads which covalently bind to CLSP (CLSP beads) were mixed with the proteins and the pull-down assay (coprecipitation experiment) was performed to examine the binding with CLSP (FIG. 8a). The concentrations of CLSP, adiponectin, annexin II, ApoE3 or ApoE4 in a mixture were all set to be 1 nM as conditions. First, when other proteins do not exist, a constant amount of adiponectin, annexin II, ApoE3, or ApoE4 was co-precipitated with CLSP beads (Lanes 2-4 and 7). Next, when co-precipitated with adiponectin, although slightly reduced, a significant amount of ApoE3 or ApoE4 was co-precipitated with CLSP beads (Lane 5 and 8). Similarly, when co-precipitated with ApoE3 or ApoE4, although slightly reduced, a significant amount of adiponectin was still co-precipitated (Lane 5 and 8). Importantly, the amount of co-precipitated ApoE was equal to or slightly larger than the amount of co-precipitated adiponectin (FIG. 10a). The addition of annexin II did not reduce the amount of ApoE3 or ApoE4 co-precipitated with CLSP (Lanes 6 and 9). In contrast, annexin II was hardly co-precipitated with CLSP beads in the presence of ApoE3 or ApoE4 (Lanes 6 and 9). These results do not indicate that adiponectin competes with ApoE for binding to CLSP to suppress the inhibitory action of ApoE (which denies the first possibility).

The dissociation constants (Kd) for the binding between CLSP and adiponectin, and between CLSP and ApoE4 were then measured (FIGS. 8b and c). For this purpose, adiponectin or ApoE4 protein was conjugated to the 96-well plates. Various concentrations of recombinant CLSP C-terminally tagged with HiBiT, a chemiluminescence producing tag, were added to the plates for simultaneous incubation. After washing, the amount of CLSP-HiBiT binding to adiponectin or ApoE4 on the wells was measured. The dissociation constant between adiponectin and CLSP or between ApoE4 and CLSP was measured to be 8.8 or 7.8 pM respectively by Scatchard analysis (FIG. 8c). This result that both are close to each other completely denied the above first possibility.

### [Adiponectin and Apolipoprotein E4 Bind to Different Subdomains of CLSP]

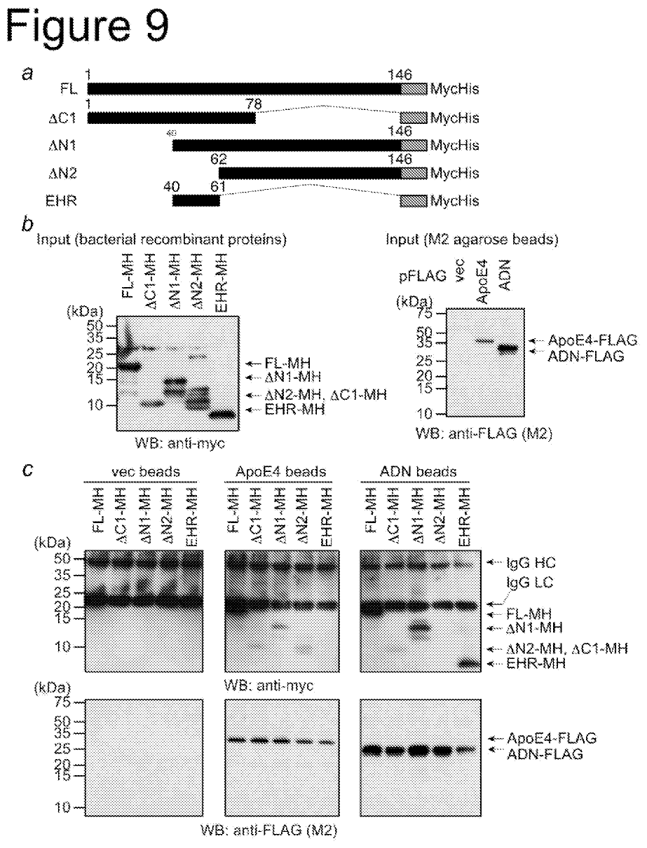

In order to investigate the second possibility, ApoE4-MycHis and adiponectin-MycHis were created and mixed with recombinant wild-type CLSP or CLSP deletion mutants created previously (5, FIG. 9a) for the pull-down assay (FIG. 9). As a result, mutants consisting of only EHR did not co-precipitate with (bind to) ApoE4 while the other 4 CLSP mutants did. On the other hand, only AN2 bound to adiponectin while the other 4 mutants did not (FIG. 9b and c). These results mean that the ApoE4-binding site of CLSP is outside the EHR and the adiponectin-binding site includes EHR. Therefore, it was revealed that adiponectin bound to the EHR to potentiate and protect the activity of CLSP, and once adiponectin bound to the EHR, the inhibitory effect of CLSP inhibiting substances caused via non-EHR regions was cancelled.

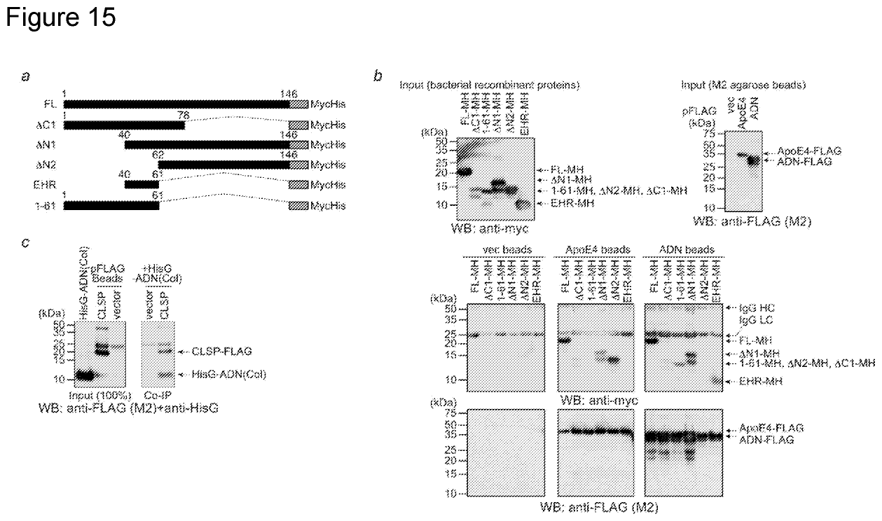

Another similar pull-down experiment found that adiponectin bound to the N-terminal region of CLSP, which consists of amino acid 1-61, while ApoE4 did not bind to the region (FIG. 15, a and b). This result, together with the result of FIG. 9, means that ApoE binds to the C-terminal region of CLSP (amino acid 62-146). The present inventors also found that CLSP bound to the so-called “collagen-homologous region (ADNCol) of adiponectin located in the central portion of adiponectin by another similar experiment (FIG. 15, c).

### [The Concentration of Adiponectin in the CSF is Markedly Reduced in AD Patients]

In order to estimate the concentration of adiponectin in the human CNS, the levels of adiponectin in the CSF obtained from autopsied AD patients and non-AD cases were measured using an adiponectin ELISA assay kit (Table 1, Table S 1, and FIG. 10). The result found that the levels of CSF adiponectin were lower in AD patients than in non-AD cases (FIG. 10b, Table 1). The mean±SEM concentration of CSF adiponectin in AD patients was 0.31±0.13 nM whereas that in non-AD cases was 0.96±0.19 nM (unpaired t-test, p=0.0065). The result is basically consistent with the result of a previous study (30), and suggests that the levels of adiponectin in the CSF of AD patients were markedly reduced. The concentrations of adiponectin in some non-AD cases are markedly lower than the mean of non-AD cases and almost equal to the mean level of AD patients.

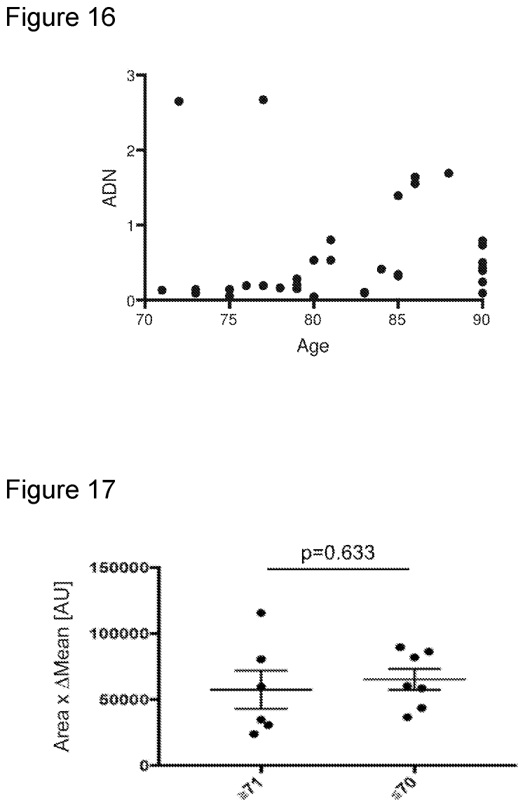

However, the average age of AD patients (78.5±0.9 years old) was significantly smaller than of non-AD cases (more than 86.3±1.4 years old) (unpaired t-test; p<0.0001 if “more than” is considered to be “equal to”) (see Table S1). Therefore, there is a possibility that the age rather than the presence of AD may affect the concentration of CSF adiponectin. In order to examine this possibility, cases aged 81-88 years old were selected from all cases in Table 1 (n=6 for AD, the average age±SEM=83.0±0.6 years old; n=5 for non-AD, the average age±SEM=85.2±1.2 years old; unpaired t-test for ages; p=0.11) (Table 2), and the CSF adiponectin levels thereof were compared. The result found that adiponectin levels are markedly downregulated in the CSF of AD patients in this age group (AD, 0.30±0.07 nM; non-AD, 1.41±0.16 nM; unpaired t-test, p<0.0001) (FIG. 10c, Table 2). It was further investigated whether or not the CSF adiponectin levels correlate with ages using data from all subjects. As expected, there was no significant correlation between age and CSF adiponectin concentration (correlation coefficient=0.0055) (FIG. 16).

### [SH3BP5, the Core Effector of Humanin/CLSP-Induced Intracellular Signaling Pathway, is Reduced in Neurons of AD Patients]

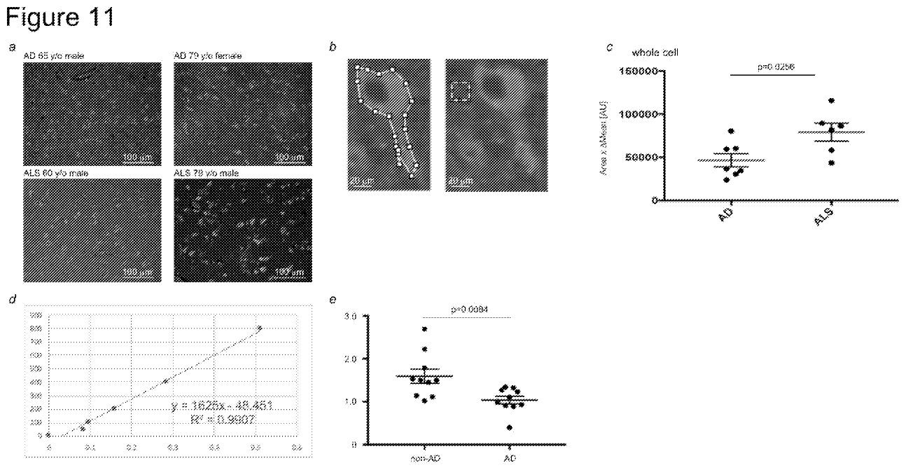

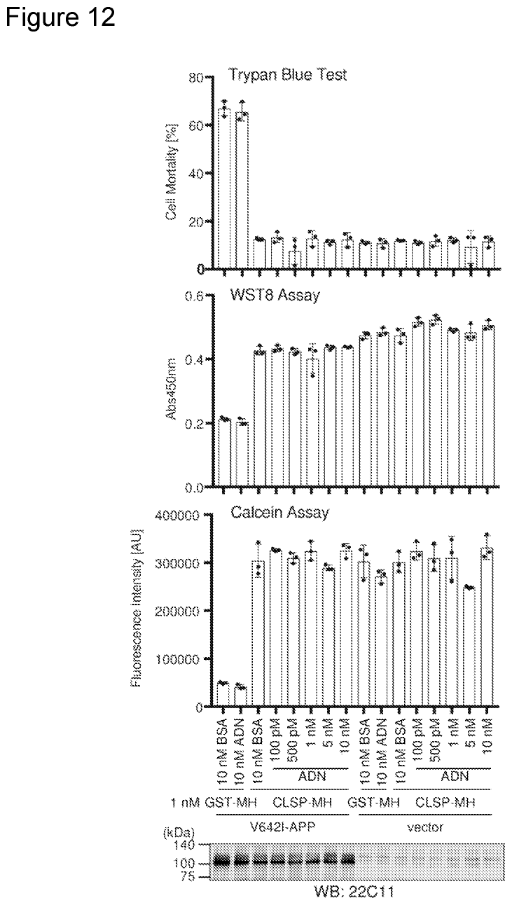

The results of FIG. 12 indicated that the amount of CLSP/adiponectin complex corresponding to the amount of active CLSP was smaller in the brains of AD patients than those of non-AD cases. In order to confirm (verify) this experimental fact, we tried to quantify the intensity of the intraneuronal CLSP-induced signaling by measuring the levels of intraneuronal SH3BP5. The reason why SH3BP5 is measured as a method for quantification is that a previous study demonstrates that SH3BP5 is the core effector of the humanin/CLSP-induced intracellular signaling via htHNR and the expression levels increase when humanin/CLSP binds to htHNR (24). In fact, when CLSP/humanin binds to htHNR, the transcription of SH3BP5 is activated via STAT3 in the study (24). The result indicates that the expression levels of SH3BP5 are elevated, and higher levels of SH3BP5 inhibit V642I-APP-induced death signals by directly forming a complex with JNK to inhibit JNK. For this purpose, first, the temporal or occipital lobes (non-motor neuronal region) of brains obtained from autopsied AD patients and amyotrophic lateral sclerosis (ALS) patients were stained immunohistochemically using the SH3BP5 antibody (Table 3 and Table S2). The brains of ALS patients were used as negative controls because the neurodegeneration occurs only in the motor neurons in the motor area of the temporal lobe and there were not abnormalities in the neurons of the temporal lobe or occipital lobe in ALS. The result of this experiment found that SH3BP5 levels were reduced in neurons of AD cortices compared with those of ALS (unpaired t-test, p=0.0256) (FIGS. 11a, b and c, Table 3). Considering that the average age of AD patients was higher than the average age of ALS patients (78 vs. 69 years old), we also examined whether or not the aging, rather than the presence of AD, is a determining factor of SH3BP5 levels. The result found that SH3BP5 levels were not significantly lower in older persons (equal to or older than 71 years old) than in younger persons (younger than 71 years old) (unpaired t-test, p=0.633) (FIG. 17).

In addition, SH3BP5 levels in tissue lysates derived from the temporal lobes of autopsied AD patients and non-AD cases were measured using SH3BP5 ELISA assays (Table 4 and Table S3). The result found that SH3BP5 levels were significantly reduced in the temporal lobes of the AD patients than those of non-AD cases (unpaired t-test, p =0.0084) (FIGS. 11d and e, Table 4).

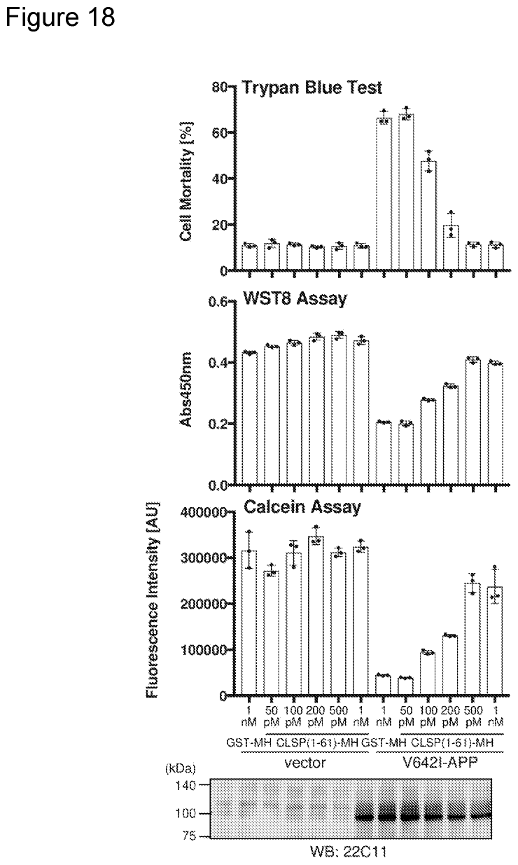

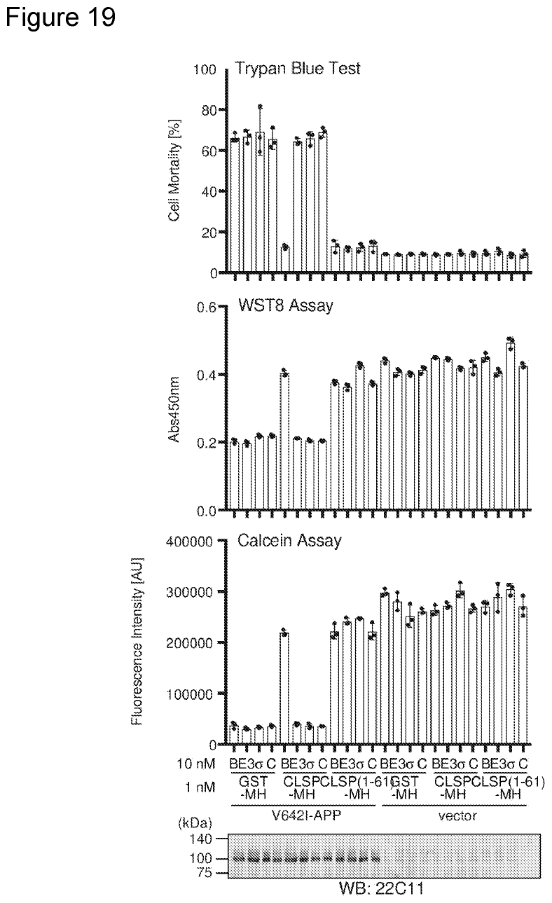

As further expansion, it was found that CLSP1-61, a CLSP derivative, did not bind to ApoE4 (FIGS. 15, a and b). Besides, there is a high possibility that CLSP1-61 includes EHR and thus retains the CLSP activity. When its cell death-suppressing activity was examined using the cell death assays, the minimal concentration that suppresses cell death was 0.5 nM (FIG. 18) and was almost equivalent to that of wild-type CLSP (FIG. 2). Furthermore, when it was examined whether or not 3 types of CLSP inhibiting substances can suppress the activity of CLSP1-61, the activity of CLSP1-61 could not be inhibited even by a 10-fold amount of each inhibiting substance (ApoE4, 14-3-3σ, calreticulin) as expected (FIG. 19). Therefore, it was proved that CLSP1-61 was insensitive to the inhibition by inhibiting substances and moreover was a CLSP derivative which keeps the activity.

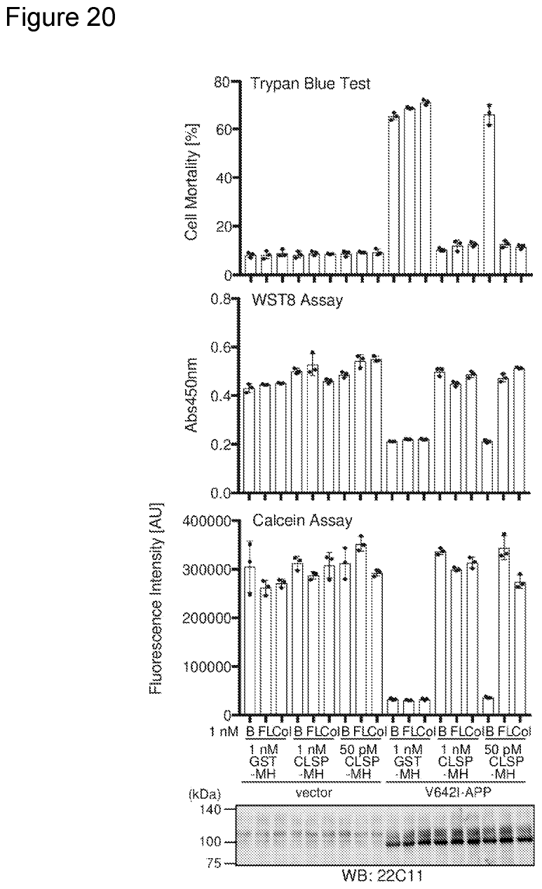

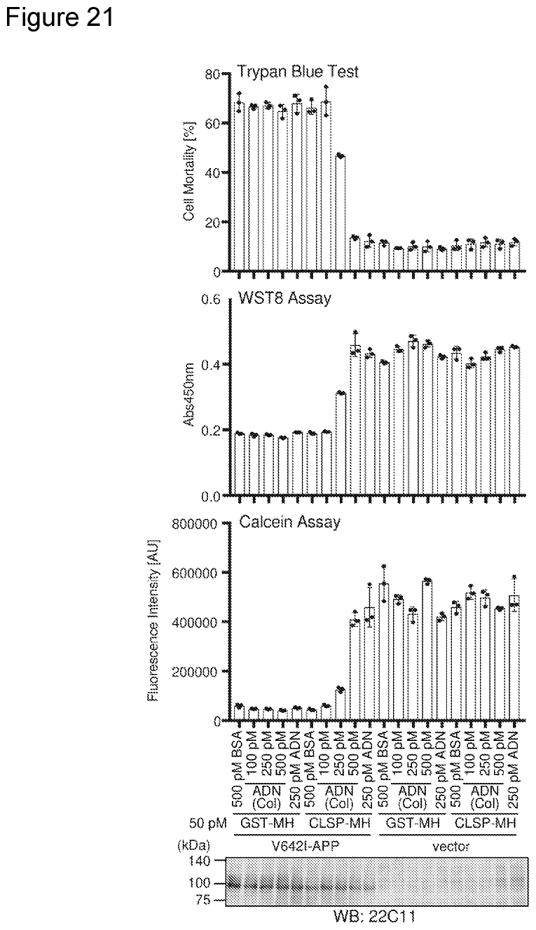

It was also found that CLSP bound to the collagen region of adiponectin (ADNCol) (FIG. 15, c). Therefore, it was examined whether or not the CLSP activity-potentiating effect of adiponectin is sufficient only by ADNCol. As a result, as is the case with 1 nM wild-type adiponectin, 1 nM ADNCol potentiated the activity of 50 pM CLSP and completely suppressed cell death (FIG. 20). Furthermore, the minimal concentration of ADNCol that provides full cell death-suppressing activity to 50 pM CLSP was found by increasing and decreasing the amount of ADNCol using the same assays. As a result, it was found that the CLSP-potentiating activity was slightly weaker than of wild-type adiponectin, the concentration was 0.5 nM (FIG. 21). Therefore, it was revealed that ADNCol had slightly weaker activity than of wild-type adiponectin but was a protein having almost the same function.

### [A Fusion Polypeptide Consisting of CLSP1-61 and the Collagen-Homologous Region of Adiponectin has Potent AD-Protecting Activity]

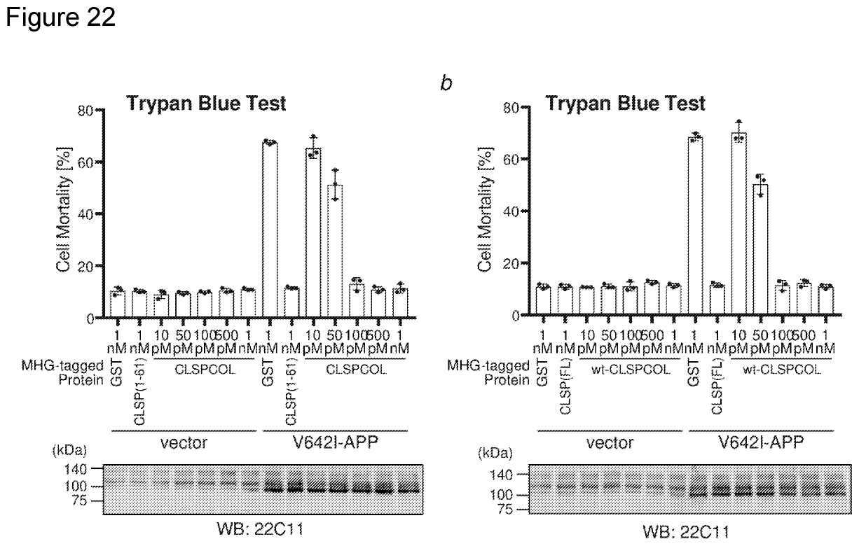

Next, two hybrid polypeptides consisting of CLSP1-61 and the collagen-homologous region of adiponectin, and wild-type CLSP and the collagen-homologous region of adiponectin (referred to as “CLSPCOL” and “wt-CLSPCOL” respectively) were prepared and it was investigated whether or not they retain both the activities of CLSP and adiponectin. Both the regions of the hybrid polypeptides are connected by peptides coding Myc tag and HisG tag (consisting of 6×histidine and glycine). CLSPCOL and wt-CLSPCOL have more potent CLSP activity than CLSP1-61 and wild-type CLSP (FIG. 18) (the minimal concentrations of both the peptides that completely suppress neuronal cell death are about 100 pM: FIG. 22). This result indicates that the functions of CLSP1-61 and wild-type CLSP are not broken by the C-terminal binding of the collagen-homologous region of adiponectin, and conversely the collagen-homologous region of adiponectin potentiates the functions of CLSP1-61 and wild-type CLSP. Therefore, it is not thought that the function of the collagen-homologous region of adiponectin is broken by the N-terminal binding of CLSP1-61.

### [CLSPCOL Passes Efficiently the Blood-Brain Barrier]

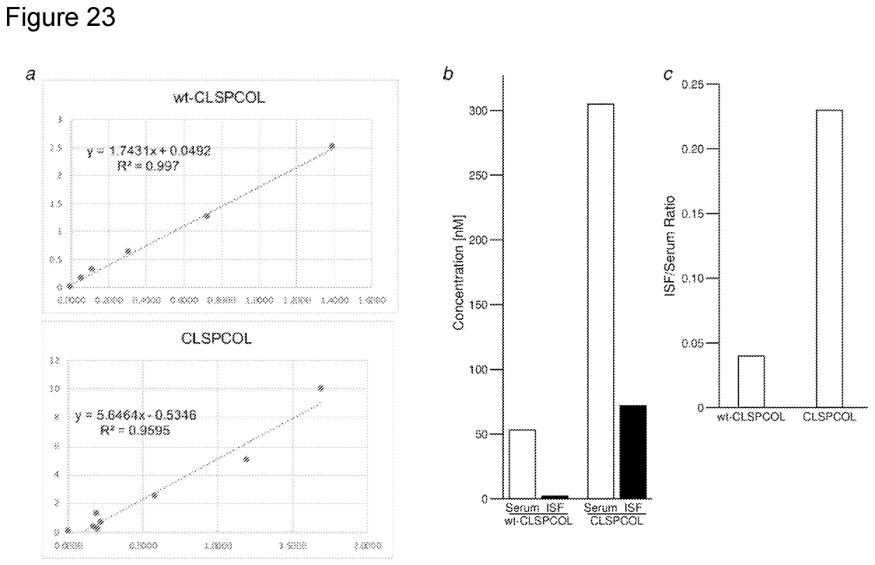

The concentrations of CLSP in the cerebrospinal fluids and serum were about 5 nM and 500 nM at an hour after the intraperitoneal injection of 5 nmol CLSP in mice (5). On the other hand, the concentration of adiponectin in human cerebrospinal fluids is 1/1000 the concentration of adiponectin in human serum (30). Therefore, it was investigated whether or not CLSPCOL and wt-CLSPCOL pass the blood-brain barrier at a rate equal to that of mouse wild-type CLSP. The concentrations of CLSPCOL in serum and interstitial fluid (ISF)-containing brain lysates were about 305 nM and 72 nM at an hour after the intraperitoneal injection of 10 nmol CLSPCOL (FIG. 23 and Table 1). The estimated concentrations of wt-CLSPCOL in serum and interstitial fluid-containing brain lysates at an hour after the injection were about 53 nM and 2.1 nM. Because the concentration of wt-CLSPCOL in ISF-containing brain lysates was less than the lower detection limit of the ELISA assay used (4.5 nM), the temporary concentration of 2.1 nM may be incorrect, whereas the concentration is certainly less than 4.5 nM. The concentration of CLSPCOL in brain lysates is estimated to be about ¼ to ⅕ the concentration in serum, while the concentration of wt-CLSPCOL is estimated to be less than 1/10 the concentration thereof in serum. Therefore, the transfer of CLSPCOL to the central nervous system is more efficient than of wt-CLSPCOL. Considering the efficiency of the transfer of CLSP from serum to the cerebrospinal fluids in the published results (5), the transfer of CLSPCOL passing the blood-brain barrier is considered to be much more efficient than of wt-CLSP.

### [CLSPCOL is not Inhibited by ApoE3 and 14-3-3σ but Mildly Inhibited by Calreticulin]

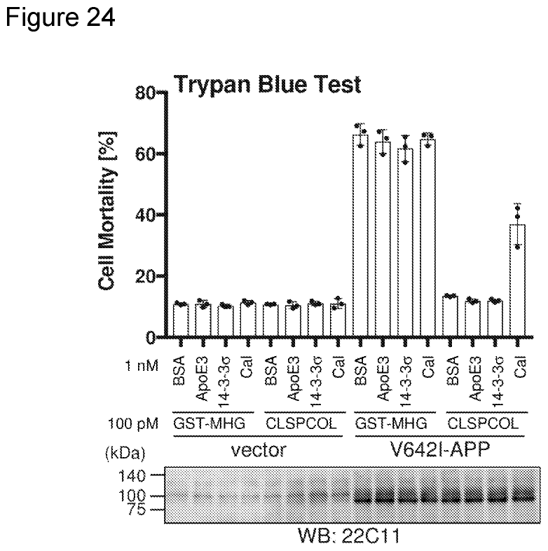

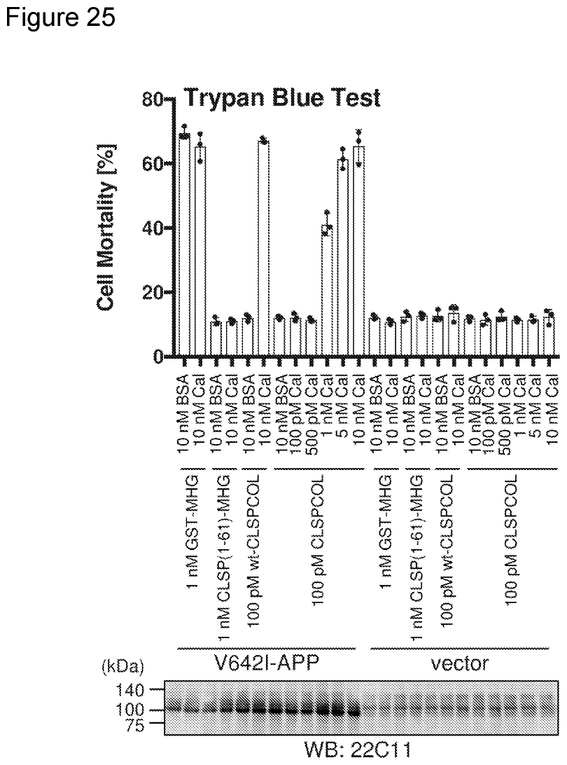

When it was examined whether or not CLSPCOL is inhibited by three types of inhibitors, it was found that it was not inhibited by 10-fold higher concentrations of ApoE3 and 14-3-3σ but unexpectedly was mildly inhibited by a 10-fold higher concentration of calreticulin (FIG. 24). On the other hand, wt-CLSPCOL was not inhibited by all the inhibitors (FIG. 25). When the concentration of calreticulin added was changed by degrees, it was found that calreticulin began to show the inhibitory activity from a concentration of 1 nM with respect to 100 pM CLSPCOL (FIG. 25).

### [Discussion]

The idea that neurodegeneration is caused by increased neurotoxic substances or the mechanism of nerve damage in neurodegenerative disorders including AD has been widely accepted. An increase in Aβ levels (aggregated fibril forms of Aβ in senile plaques and/or soluble Aβ oligomers) has been regarded as the primary insult in AD for 20 years or longer (2). Besides, a possibility that the hyperphosphorylated tau, and the mechanism of nerve damage related to amyloid β precursor protein and presenilins, which are not directly related to an increase in Aβ levels, may be involved as toxicity has been described. In addition, a previous study and the present study presented a possibility that, in addition to these well-known mechanisms of nerve damage, a reduction and attenuation of the AD-protecting factors will contribute to the development of AD. Among these, CLSP is presumed to likely play a core role as the AD-protecting (defensing) factor (6).

Humanin and CLSP inhibit AD-related neuronal cell death in vitro (5, 6). In addition, CLSP suppresses synaptic loss and memory impairment without relation to regulation of Aβ in AD model mice (8). The result of the latter is also supported by a series of previous studies (25-27). That is, the studies are that a potent derivative of humanin, another antagonist of htHNR, suppressed memory impairment in AD model mice. Considering these results, the present inventors have proposed a hypothesis on the AD pathogenesis that two events were necessary for the onset of AD; an increase in the AD-related neurotoxicity and a decrease in the AD-protecting activity. If this hypothesis is correct, because neuronal cell death (and dysfunction) cannot be caused in the presence of a sufficient concentration of active CLSP even if the AD-related neurotoxicity is sufficiently potentiated, AD does not appear. In addition, when there is not sufficient AD-related neurotoxicity, neuronal cell death (and dysfunction) is not caused (i.e., AD does not appear) even when the effect of CLSP is reduced. The experimental results that the levels of adiponectin and SH3BP5 in the CNS of some “non-AD” controls in addition to almost all AD cases among the data shown in the present examination are reduced (FIGS. 10 and 11), support that these ideas are extremely valid.

CLSP is considered to bind to the heterotrimeric humanin receptor and be a core AD-protecting factor that activates STAT3-induced survival signaling pathway (5, 6 and 8), and the abnormal regulation thereof likely contributes to AD pathogenesis. Considering the concentrations and activity of a plurality of CLSP inhibiting substances observed in the present invention, ApoE is considered to be a core inhibiting substance (FIG. 2). The concentration of total ApoE is estimated to be much higher than the concentration of CLSP in the human CNS (18-20) (14). Therefore, if an in vivo CLSP activity-regulated model that is composed of only CLSP and much higher amounts of CLSP inhibitors is correct, the AD-protecting activity is likely almost null in vivo.

As described by the present invention, however, if adiponectin potentiates the CLSP activity and protects CLSP from CLSP inhibitors in a dominant fashion by binding to the endogenous humanin-homogeneous region (EHR) of CLSP (FIGS. 5 to 7), the in vivo CLSP activity is guaranteed even in the presence of higher concentrations of the CLSP inhibiting substances. Even in the presence of much higher concentrations of CLSP inhibitors, 0.2-0.25 nM adiponectin can completely keep the CLSP (1 nM) activity (FIGS. 5 and 7). In the case of non-AD, the concentration of adiponectin in the CSF is 0.96±0.19 nM (FIG. 10 and Table 1), and consequently the CLSP activity is likely kept.

Adiponectin exerts a variety of metabolic functions including glucose and lipid metabolisms in peripheral tissues (28). It increases insulin signaling, anti-inflammatory, anti-oxidative and anti-atherogenic functions possibly via two canonical adiponectin receptors on the cell membrane. The transfer of adiponectin through the blood-brain barrier appears to be very limited. The concentration of adiponectin in the CSF is nearly 10³-fold lower than the concentration in serum (29, 30). Given the presence of the canonical adiponectin receptors in the CNS, adiponectin functions in the CNS as a regulator of glucose metabolism and a neurogenesis enhancer, and is hypothesized to function, for example, as a protective factor against ischemic brain damage (31). Many studies have provided evidence that the insufficiency of adiponectin or the abnormal regulation of adiponectin signaling is linked to the onset of AD (31). The increase in serum adiponectin levels (29, 30) may be an independent risk factor of AD (32). A study, meanwhile, indicated that AD-like pathology appeared in significantly many type II diabetic patients having a low concentration of serum adiponectin (33). Adiponectin levels are downregulated in the CSF of AD patients and inversely correlated with the increase in Aβ levels (30). Adiponectin knockout mice show an AD-like pathology (34).

The present invention indicated that the concentration of adiponectin was reduced in the CSF of AD patients (FIG. 10). This result is consistent with the previous report (30). Considering that the protein concentrations in the CSF are correlated with the concentrations in brain interstitial fluids (36), these results suggest that the concentration of adiponectin was reduced in the intestinal fluids in the AD brains and may be incapable of keeping the CLSP activity. As actual data, the mean±SEM concentration of CSF adiponectin in AD patients was 0.31±0.13 nM whereas that in non-AD cases was 0.96±0.19 nM (unpaired t-test, p=0.0065) (FIG. 10 and Table 1). As shown in FIGS. 5 and 7, the minimal concentration of adiponectin in the perineural site required to keep CLSP fully active is estimated to be 0.20 to 0.25 nM, which is near the reduced mean concentration of CSF adiponectin in AD patients. A possibility is suggested that a sufficient amount of adiponectin exists in the perineural site of non-AD whereas the amount of adiponectin in the site of AD is insufficient. As further data supporting this idea, it was found that the intraneuronal levels of SH3BP5, which is a main mediator of humanin/CLSP signals, were reduced in AD cortices (FIG. 11). As similar data, a previous study has already indicated that the levels of STAT3 with the phosphorylated 705th tyrosine, an active form of STAT3 which is activated by humanin/CLSP signals, were reduced in the hippocampal neurons of AD patients (35). It is indicated that humanin and CLSP bind to htHNR and activate intraneuronal signaling mediated by STAT3 (6) and SH3BP5 (24) to function as the AD-protecting factor.

Previous studies also indicate data that adiponectin increases in the serum of AD patients (29, 30) but is reduced in AD brains (FIG. 10 and Table 1 and Table 2) (30). One interpretation of this finding is the idea that adiponectin levels are reduced by one or some of AD-related abnormalities caused in the central nervous system, the production of adiponectin in the adipose tissue is secondarily upregulated to recover the insufficiency, and consequently the levels are increased in serum. In fact, the previous study has suggested that adiponectin may be reduced in the central nervous system of AD patients because adiponectin is immobilized into intraneuronal neurofibrillary tangles containing hyperphosphorylated tau (30). If this idea is correct, the increase in Aβ and the accumulation of hyperphosphorylated tau, a downstream target thereof, are regarded as the main causes of a reduction in adiponectin levels in AD. On the other hand, another idea is that a reduction in adiponectin levels in the central nervous system is caused because the transfer of adiponectin through the blood-brain barrier in AD is impaired. Although a large amount of evidence which indicates that the function of the blood-brain barrier is impaired in the brains of AD has been presented (37) until now, there has not existed the data about the transfer of adiponectin through the blood-brain barrier, and thus it is still unknown which idea is correct as of this moment.

ApoE4 is a major risk factor for AD. The mechanism underlying the ApoE4-mediated increase in the onset of AD has been extensively investigated by many studies until now. ApoE4 is considered to exert neurotoxicity by multiple gain-of-function and loss-of-function mechanisms in both Aβ-dependent and independent fashions (38). A study has been well known that among these, the production of Aβ, the clearance of Aβ from central nerves, and the aggregation of Aβ are affected by intracellular information mediated by the ApoE receptors and these phenomena move toward the onset of AD in ApoE4 carriers.

The present invention indicated that ApoE4 was a slightly stronger CLSP-inhibiting substance than ApoE3 (FIGS. 2b and c). Considering much higher concentrations of ApoE than the concentration of CLSP in the CNS, slight differences have no significance and ApoE3 and ApoE4 likely reduce the CLSP activity similarly. However, given the assumption that only free ApoE that is not lipidated (or not recruited into high density lipoprotein-like lipid particles) may be able to suppress CLSP, when the concentration of non-lipidated ApoE is comparable to the concentration of CLSP, a slight difference in the CLSP-inhibiting effect of ApoE can become a determinant of the onset in the state that adiponectin levels are reduced (persons affected with AD). In this case, since ApoE4 suppresses CLSP activity more strongly (FIGS. 2b and c), ApoE4 gene carriers become more susceptible to AD insults than non-ApoE4 carriers. Unfortunately, there are no specific methods to measure the amount of non-lipidated ApoE to examine the validity of this idea.

The present invention presumed that intraneuronal CLSP signaling was reduced in AD brains by the finding that the adiponectin levels are reduced in the CSF of AD patients and the levels are close to the limit level that can protect CLSP (0.3 nM) (FIG. 10 and Table 1 and Table 2), and the finding that the levels of SH3BP5 and activated STAT3 are reduced in AD brains (FIG. 11) (35).

In the present invention, however, since it is technically almost impossible to measure the concentration of adiponectin in the interstitial fluid around AD brain neuronal cells, the concentration in CSF, which is close to that in the interstitial fluid, is measured and events in the interstitial fluid are discussed based on the concentration.

Furthermore, since it is technically impossible to directly show that intraneuronal CLSP-induced signals are reduced in AD brains, that is indirectly shown in the present invention. Since SH3BP5 and STAT3 are simultaneously regulated by signaling pathways induced by bioactive substances centered on various cytokines, a reduction in SH3BP5 levels and the inactivation of STAT3 can occur without a decrease in CLSP-induced signaling. Therefore, it cannot be determined that CLSP-induced signals are reduced by both the reductions. However, it is well known that inflammation commonly occurs in the CNS of AD and consequently various inflammatory cytokines are produced. Various elevated inflammatory cytokines increase the levels of intraneuronal activated STAT3 and SH3BP5 (a downstream target of STAT3). Therefore, when the levels of activated STAT3 and SH3BP5 in neurons of AD are lower than normal, considering that there are elevations by various inflammatory cytokines released in the vicinity, the idea that the reductions in activated STAT3 and SH3BP5 indicate a reduction in CLSP-induced signals appears valid.

### [CLSPCOL]

CLSPCOL is free from the inhibition by the CLSP inhibitors and has potent AD-protecting activity (FIG. 22). Furthermore, the collagen-homologous region (COL) of adiponectin retains the activity to potentiate and protect endogenous wild-type CLSP. Furthermore, CLSPCOL penetrates the blood-brain barrier efficiently (FIG. 23). Therefore, the fusion proteins of the present invention such as CLSPCOL do not have obvious weak points currently and can be an AD-agent candidate which can be delivered via a peripheral route.

However, the mechanism that CLSPCOL passes the blood-brain barrier more efficiently than wt-CLSPCOL and CLSP has not been sufficiently clarified. Since there is an obvious difference in efficiency between CLSPCOL and wt-CLSPCOL (FIG. 23 and Table L1), the deletion of the C-terminal domain (amino acid 62-146) of CLSP likely promoted efficiency. That is, the C-terminal half of CLSP may include a region which inhibits the transfer through the blood-brain barrier. In addition, efficiency is likely increased by the attachment of the collagen-homologous region of adiponectin.

CLSPCOL was mildly inhibited only by calreticulin of inhibiting substances (FIGS. 24, 25). Although the specific mechanism is unknown, possibly a region including an artificially created fusion portion is presumed to have an affinity for calreticulin. However, it is expected that the inhibitory effect is weak and also the concentration of calreticulin in the central nervous system is lower than that required to show the inhibitory effect (less than 1 nM), and thus it is thought that there are not problems in actual clinical application.

The materials and methods used in Examples above are as follows. Unless otherwise specified, appropriate methods and means known by those skilled in the art were used.

### [Genes and Vectors]

The human CLSP was inserted in pcDNA3.1-MycHis (Invitrogen, Carlsbad, Calif.) to express human CLSP-MycHis, C-terminally tagged with MycHis, (CLSP-MycHis) in mammalian cells (5). Human apolipoproteins E3, E4, adiponectin, annexin II, and annexin V cDNAs were inserted into pHA vector, a CMV promoter-driven expression vector having a C-terminal hemagglutinin A (HA)-tag. Mouse V642I-APP cDNAs inserted into the pcDNA3.1/MycHis vector are described in previous literature (5). Apolipoproteins E3, E4 and adiponectin cDNAs were inserted into the pFLAG vector, which was used as a C-terminally FLAG-tagged protein expression vector.

Schistosoma japonicum glutathione S-transferase (GST)-tagged recombinant proteins were generated in bacteria using the pGEX vector (Promega, Madison, Wis.) as described in literature (5). For the generation of C-terminally HiBiT-tagged CLSP, a sense (SEQ ID NO:4):

(5′-CCCGGGGTGAGCGGCTGGCGGCTGTTCAAGAAGATTAGCTGAGAAT
TC-3′),

and
an antisense (SEQ ID NO:5):

(5′-CCCGGGGTGAGCGGCTGGCGGCTGTTCAAGAAGATTAGCTGAGAAT
TC-3′),

oligonucleotides encoding the HiBiT (VSGWRLFKKIS) amino acid sequence were in vitro annealed and inserted into the pGEX-2T-CLSP plasmid at the SmaI-EcoRI site.

A full-length human adiponectin cDNA in the pCMV-SPORT6 vector was purchased from Invitrogen (cat. no.: 6192794, CA). To make recombinant N-terminally GST-tagged proteins that were C-terminally tagged with MycHisG, the sequence of the pGEX2T-MycHis vector was mutated using KOD-Plus-Mutagenesis Kit (cat. no.: SMK-101, TOYOBO CO., LTD., Tokyo, Japan) with mutagenesis primers below to cause the C-terminal addition of a glycine residue.

Sense primer (SEQ ID NO: 6):
(5′-GGTTGAGAATTCATCGTGACTGACTGACGATCTGCCTCGCG
CG-3′),
and
antisense primer (SEQ ID NO: 7):
(5′-ATGATGATGATGATGATGATCCTCTTCTGAGATGAGTTTTT
G-3′).

A cDNA of the collagen-homologous region of human adiponectin was amplified by KOD DNA polymerase (cat. no.: KOD-101, TOYOBO CO., LTD., Tokyo, Japan).

Sense primer (SEQ ID NO: 8):
(5′-GGATCCATGAGAGGATCGCATCACCATCACCATCACGGGT
CC-3′),
and
antisense primer (SEQ ID NO: 9):
(5′-GAATTCTCAAGGTTCTCCTTTCCTGCCTTGGATTCCCGGA
AAGC-3′).

The amplified cDNA was subcloned into the pQE30 vector (QIAGEN, Tokyo, Japan) at the BamHI-EcoRI site.

A cDNA of the collagen-homologous region of adiponectin, N-terminally tagged with MycHisG, was amplified by LA Taq polymerase (TaKaRa, cat. no.: RROO2A, Tokyo, Japan).

Sense primer (SEQ ID NO: 10):
(5′-AAGCTTGAACAAAAACTCATCTCAGAAGAGGATCATCATCATCATC
ATCATGGTATGGGGCATCCGGGCCATAATGGGGCCCCAGGCC-3′)
and
antisense primer (SEQ ID NO: 11):
(5′-GAATTCTCAAGGTTCTCCTTTCCTGCCTTGGATTCCCGGAAAG
CC-3′).

The amplified cDNA was subcloned into the pGEX-2T-CLSP and -CLSP(1-61) plasmids to obtain wt-CLSPCOL and CLSPCOL consisting of the collagen-homologous region of CLSP-MycHisG-adiponectin and the collagen-homologous region of CLSP(1-61)-MycHisG-adiponectin respectively.

### [Recombinant Proteins]

GST-human CLSP, C-terminally tagged with MycHis, (GST-CLSP-MycHis) was expressed in E. coli BL-21 at 37° C. for 6 hours in 1 mM isopropyl-thio-β-D-galactopyranoside. GST-CLSP-MycHis was bound to glutathione sepharose (GE Healthcare) and the CLSP-MycHis portion was released from the glutathione sepharose by co-incubation in PBS containing thrombin (1 unit/ml) (cat. no.: T6634-100 UN, Sigma-Aldrich, St. Lois, Mo.) at 25° C. overnight as described in literature (14). Recombinant CLSP deletion mutants, C-terminally labeled with MycHis (5), and GST-CLSP-HiBiT were produced in the same way. Recombinant annexins II, V and SH3BP5 proteins were also made similarly from GST-annexin II, annexin V and SH3BP5 produced in bacteria. Recombinant GST-MycHis and GST-human 14-3-3σ were expressed in E. coli BL-21 at 37° C. for 6 hours in 1 mM isopropyl-thio-β-D-galactopyranoside, bound to glutathione sepharose, released from the glutathione sepharose by co-incubation in the presence of 50 mM glutathione, and dialyzed in PBS. The collagen-homologous region of adiponectin, N-terminally tagged with 6×HisG, was expressed in E. coli M15 [pREP4] (Qiagen) at 37° C. for 4 hours in 1 mM isopropyl-thio-β-D-galactopyranoside, bound to Talon Metal Resin (Clontech, Palo Alto, Calif., USA), and purified according to the manufacturer's instruction. The eluted recombinant 6×His proteins were desalted by Zeba Desalting Column (Pierce) and then a one-tenth volume of 10×PBS was added to the desalted protein solution.

Recombinant human ApoE3 and ApoE4 were purchased from PeproTech (Rocky Hill, N.J.) (cat. no.: 350-02 and 350-04). Human adiponectin and trimeric adiponectin were purchased from BioVendor (Czech Republic) (cat. no: RD172029100 and RD172023100).

### [Cell Death Assays]

Neuronal cell death assays related to AD were first performed by Yamatsuji et al. (39). SH-SYSY cells were grown in DMEM/Ham F12 mixture (DMEM/F12) containing 10% FBS. SH-SYSY cells were seeded in 6-well plates at 2×10⁵/well for 12 to 16 hours, transfected with indicated vectors in the absence of serum for 3 hours, and then cultured in DMEM/F12-10% FBS with/without CLSP and/or CLSP modifiers (substances affecting CLSP). At 24 hours after the transfection, the media were replaced with DMEM/F12 containing N2 supplement (Invitrogen, Carlsbad, CA) with/without CLSP and/or CLSP modifiers. At 48 hours after the onset of the transfection, the cells were harvested to perform the cell viability (survival rate) assays using the WST-8 cell death assay kit (Dojindo, Kumamoto, Japan) or staining with calcein AM (Dojindo, Kumamoto, Japan), and the trypan blue exclusion cell mortality assays. Transfection efficiency in SH-SYSY cells was approximately 80%. All cell death experiments were performed in N=3.

### [Antibodies]

Rabbit polyclonal antibodies were produced against the N-terminal 16 amino acid peptide of human CLSP conjugated with keyhole limpet hemocyanin, and affinity-purified using immunopeptides (hCLSP-N antibody). Rabbit polyclonal antibodies against GST-CLSP-MycHis were created by immunization with recombinant GST-CLSP-MycHis (GST-CLSP antibody) produced in bacteria (5). Furthermore, antibodies were affinity-purified from crude serum using recombinant CLSP-MycHis. Affinity purification was performed using 14-3-3σ. A sigma-C antibody was produced by immunizing rabbit with the C-terminal 16 amino acid peptide of human 14-3-3σ and further affinity-purified. A polyclonal antibody against SH3BP5 (named “SH3BP5 antibody”) was produced in rabbit, affinity-purified using GST-14-3-3σ and GST-SH3BP5, and further affinity-purified using GST-SH3BP5.

Ready-made antibodies against the peptides and proteins used in the present invention were purchased from the following companies: horseradish peroxidase-conjugated FLAG epitope (clone M2, cat. no.: 158592-1MG), Sigma-Aldrich; APP (22C11, cat. no.: MAB348 (registered trademark)), Chemicon (Temecula, Calif.); Myc epitope (cat. no.: R950-25), Invitrogen (Carlsbad, Calif.); peroxidase-conjugated HA (hemagglutinin A) epitope (clone 3F10, cat. no.: 2013819), Roche Diagnostics (Alameda, Calif.); SH3BP5 antibody (Sab; cat. no.: sc-135617), Biotechnology (Santa Cruz, Calif.); SH3BP5 monoclonal antibody (clone 1D5, cat. no.: H00009467-M02), Abnoba, (Taipei, Taiwan); and HisG monoclonal antibody (cat. no.: R940-25), Invitrogen (Carlsbad, Calif.).

### [Immunoblot Analysis]

Cells were washed twice with PBS and suspended in 50 mM HEPES (pH 7.4), 150 mM NaCl, 0.1% NP-40, and Protease Inhibitor Cocktail Complete (Roche Diagnostics, Alameda, Calif.). After freezing and thawing twice, the cell lysates were centrifuged at 15,000 rpm for 10 minutes at 4° C. The supernatant and pulled-down precipitates were subjected to analysis with standard or Tris-Tricine SDS polyacrylamide gel electrophoresis (SDS-PAGE) and immunoblot analysis. Ten μg of cell lysates per lane were used for direct immunoblot analysis (5). The endogenous wild-type APPs with various lengths were simultaneously detected by the immunoblot analysis using the APP antibody to detect exogenously expressed V642I-APP. It should be noted that due to unknown reason, the detection of endogenous wild-type APPs with various lengths was not uniform among different experiments.

### [Pull-Down Analysis]

Conjugation of a recombinant protein to cyanogen bromide-activated sepharose 4B was performed according to the manufacturer's instruction (Amersham Pharmacia Biotech, Uppsala, Sweden). Briefly, 5 mg of a recombinant protein was incubated with 3 ml of cyanogen bromide-activated sepharose 4B in a coupling buffer (0.1 M NaHCO₃ containing 0.5 M NaCl, pH 8.3) at 4° C. overnight with constant rotation. Recombinant protein-conjugated sepharose was then incubated in a blocking buffer (0.2 M glycine, pH 8.0) for 2 hours at room temperature to eliminate non-specific binding. After blocking, the sepharose was washed with the coupling buffer, and 0.1 M sodium acetate buffer (pH 4) containing 0.5 M NaCl. Conjugated sepharose 4B was stored in the coupling buffer at 4° C.

Lysates from cells overexpressing various proteins were mixed with GST-MycHis or CLSP-MycHis-conjugated sepharose 4B at 4° C. overnight, followed by exhaustive washing. The pulled-down precipitates and the cell lysates were then subjected to SDS-PAGE and immunoblot analysis or staining with silver (Wako Pure Chemical, Tokyo, Japan) to examine the binding between CLSP and proteins.

In an experiment, recombinant CLSP, one of its deletion mutants (ΔN1, ΔN2, ΔC1 and EHR), C-terminally tagged with MycHis, were produced in bacteria and purified.

They were mixed with F11 cell-derived lysates containing apolipoprotein E4 or adiponectin, C-terminally tagged with FLAG, at 4° C. overnight, followed by exhaustive washing. The washed pulled-down precipitates and the cell lysates were then subjected to SDS-PAGE and immunoblot analysis.

[Preparation of Brain Lysates from Mice as Interstitial Fluid-Containing Brain Samples After the Intraperitoneal Injection of Recombinant Protein]

All experiment procedures were approved by the Institutional Animal Care and Use Committee of Tokyo Medical University. To male ICR mice (8 weeks old) purchased from Oriental Yeast Co., Ltd. (Tokyo, Japan), 10 nmol GST-MycHisG protein, CLSPCOL or wt-CLSPCOL in PBS was intraperitoneally injected as negative controls. At an hour after the injection, the mice were anesthetized with diethyl ether (Wako Pure Chemical, Tokyo, Japan). Blood was then aspirated from hearts and centrifuged at 4000×g for 10 minutes at 4° C. The vascular space of the brain was washed to remove blood by perfusing 20 ml of ice-cold lactated-Ringer's solution (Otsuka Pharmaceutical Co., Ltd., Tokyo, Japan) through the left ventricle of the heart. Subsequently, the mice were decapitated and the brains were removed. The whole brain was washed once with the lactated-Ringer's solution to remove the contamination of CSF. The brain was then homogenized in the presence of two-fold weight of saline. After the lysates were centrifuged at 4000×g for 10 minutes at 4° C., the supernatant was collected as the interstitial fluid-containing brain sample (36).

### [Human Cerebrospinal Fluid and Temporal Lobe Samples]

Postmortem CSF and temporal lobe samples from AD patients and controls were obtained from The Kathleen Price Bryan Brain Bank at the Division of Neurology, Duke University Medical Center (Tables 1 and 3). Pathological staging was performed under “the Consortium to Establish a Registry for AD” (CERAD) staging system for senile and neuritic plaques (40) and under the Braak staging systems for neurofibrillary tangles (41). All possible AD cases regarded by the CERAD staging were counted as AD cases. This study was also approved by the Ethics Committee of The Kathleen Price Bryan Brain Bank of Duke University Medical Center and the Research Ethics Committee of Tokyo Medical University.

### [Measurement of Dissociation Constants]

The dissociation constant for the binding between ApoE4 (or adiponectin) and CLSP was measured using Nano-Glo HiBiT Extracellular Detection System (Promega, cat. no.: N2420) in accordance with the instruction. For the coating of recombinant ApoE4 or adiponectin, 100 μl of 50 mM carbonate buffer (pH 9.6) containing 20 pM ApoE4 or adiponectin was incubated overnight at 4° C. in the wells of 96-well plates (Fluorescence-use Black-type Plate H, cat. no.: MS-8596KZ, Sumitomo Bakelite Co., Ltd., Tokyo, Japan). The protein-coated plates were washed three times with 200 μl of PBS. Then, 150 μl of PBS containing 1% skim milk (GIBCO) was added to each well. They were incubated for an hour at room temperature without shaking. After the plates were washed three times with 200 μl of PBS, the concentration of 100 μl CLSP-HiBiT in PBS was added to each well. The plates were further incubated overnight at 4° C. without shaking and then washed 5 times with PBS containing 0.1% NP-40, followed by the addition of 100 μl of PBS. Then, the substrate for HiBiT in the kit was added to each well. Resulting chemiluminescence was measured for each well using Wallac ARVO™ X5 (Perkin Elmer). The concentration of CLSP-HiBiT was estimated for each well, referring to a standard line that was simultaneously created by measuring chemiluminescence of wells that were filled with 100 μl of PBS containing stepwise increasing concentrations of CLSP-HiBiT. This experiment was performed in N=2.

### [ELISA]

Ready-made ELISA kit for human adiponectin was purchased from Sekisui Medical Co., Ltd. (cat. no.: 376405, Tokyo, Japan), and was used to measure the concentration of CSF adiponectin in accordance with the manufacturer's instruction. For 14-3-3σ ELISA and SH3BP5, 100 μL of 50 mM carbonate buffer (pH 9.6) containing 0.6 μg/ml GST-sigma antibody or 1 μg/ml SH3BP5 monoclonal antibody (clone 1D5, cat. no.: H00009467-M02, Anoba, Taipei, Taiwan)) was incubated overnight at 4° C. in the 96-well plates (ELISA Plate H, cat. no.: MS-8896FZ, Sumitomo Bakelite Co., Ltd., Tokyo, Japan). The capture antibody-coated plates were washed three times with 400 μl of a wash buffer (PBS containing 0.1% NP40) in each well and filled with 300 μl of PVDF Blocking Reagent (TOYOBO cat. no.: NYPBRO1, Tokyo, Japan) for an hour at room temperature without shaking. After washed three times with 300 μl of the wash buffer, the plates were filled with a 100 μl solution of stepwise increasing concentrations of recombinant 14-3-3σ or SH3BP5 in PBS solution (for the measurement of a standard curve). Human CSF samples or human temporal lobe lysates were incubated for 2 hours at room temperature with shaking at 250 rpm. Then, the plates were washed with 300 μl of the wash buffer. The peroxidase-labeled sigma-C antibody or SH3BP5 antibody was prepared using as a detection antibody Ab-10 Rapid Peroxidase Labeling Kit (Dojindo, cat. no.: LK33, Kumamoto, Japan) or Peroxidase Labeling Kit-HN₂ (Dojindo, cat. no.: LK11, Kumamoto) respectively. To each well, 100 μl of 1.0 μg/ml detection antibody in Can Get Signal Solution 2 (TOYOBO cat. no.: NKB_301) was added, and the plates were incubated for an hour at room temperature with shaking (250 rpm). After washed 5 times with 300 μl of the wash buffer, R & D TMB Substrate solution (R & D Systems, cat. no.: DY999) was added to each well, and the plates were incubated for 10 minutes at room temperature. The reaction was stopped by adding 50 μl of H₂SO₄. Absorbance at 450 nm was measured using Wallac ARVO™ X5 (Perkin Elmer).

Biotin-labeled anti-HisG antibody was prepared using biotin-labeled Kit-NH₂ (Dojindo, cat. no.: LK03, Kumamoto, Japan) in accordance with the manufacturer's instruction. In the case of ELISA for CLSPCOL and wt-CLSPCOL (including Myc and HisG tags in series as binding peptides), 100 μl of 50 mM carbonate buffer (pH 9.6) containing 25 μg/ml CLSP-N antibody (capture antibody) was incubated overnight at 4° C. in the 96-well plates (ELISA Plate H, cat. no.: MS-8896FZ, Sumitomo Bakelite Co., Ltd., Tokyo, Japan). The capture antibody-coated plates were washed three times with 400 μl of the wash buffer (PBS containing 0.1% Tween 20) in each well, and filled with 300 μl of PVDF Blocking Reagent (TOYOBO cat. no.: NYPBRO1, Tokyo, Japan), and retained for an hour at room temperature without shaking. After washed three times with 300 μl of the wash buffer, the plates were filled with 100 μl of PBS containing stepwise increasing concentrations of GST-MycHis, CLSPCOL and wt-CLSPCOL (for the measurement of a standard curve), or mouse brain lysates and incubated for 2 hours at room temperature. After washed with 300 μl of the wash buffer, the plates were filled with 100 μl of 1000-fold diluted biotin-conjugated anti-HisG antibody in Can Get Signal Solution 1 (TOYOBO cat. no.: NKB-201), and incubated. The plates were incubated for an hour at room temperature. Then, the plates were washed three times with 300 μl of the wash buffer. Then, they were filled with 100 μl of 2000-fold diluted streptavidin-conjugated HRP (Invitrogen) in Can Get Signal Solution 2 (TOYOBO cat. no.: NKB-301), and incubated for an hour at room temperature. After washed 5 times with 300 μl of the wash buffer, the plates were filled with 100 μl of R & D TMB Substrate solution (R & D Systems, cat. no.: DY999), and incubated for 3 minutes at room temperature. The reaction was stopped by adding 50 μl of H₂SO₄. Absorbance at 450 nm was measured using Wallac ARVO™ X5 (Perkin Elmer).

### [Immunohistochemical Analysis of Human Samples]

This study was approved by the Institutional Human Ethics Committees of Tokyo Medical University. Histological brain samples were obtained at Gunma Geriatric Research Hospital under established procedures after obtaining a written informed consent from the family of each patient. Patients were diagnosed as having AD using clinical criteria and the diagnosis was confirmed by neuropathological analysis at autopsy. At autopsy, brains were fixed with 4% paraformaldehyde in PBS (pH 7.4), embedded in paraffin, and then subjected to neuropathological examination. Cerebral cortices and hippocampi used in this study were obtained from samples of 6 patients with sporadic AD and 5 patients with sporadic amyotrophic lateral sclerosis (ALS), a representative motoneuron-specific neurodegenerative disease.

Sliced sections were deparaffinized, rehydrated to PBS, and unmasked in ANTIGEN UNMASKING SOLUTION (Vector Laboratories, Burlingame, Calif.) for 15 minutes. Subsequently, the sections were incubated at room temperature for 20 minutes in a blocking solution containing goat normal serum and 0.3% Triton X-100 in TBS, and then incubated at 4° C. for 3 overnights with 5 μg/ml mouse IgG1 as a negative control (R & D Systems cat. no.: MAB002, Minneapolis, Minn.) or SH3BP5 (Sab) monoclonal antibody clone PL-A23 (Santa Cruz Biotechnology, cat. no.: sc-135617, Santa Cruz, Calif.) in PBS containing 1% BSA. Immunoreactivity was visualized using TSA (Tyramide Signal Amplification)-Plus Fluorescein System (Perkin-Elmer, Waltham, Mass.) (Tyramide-Red method). Fluorescence-labeled samples were observed with a fluorescence microscope (Biozero, KEYENCE, Osaka, Japan). Fluorescence images were analyzed using NIH Image J 1.37v.

### [Quantification of SH3BP5 Immunofluorescence Intensity in Neurons]

Using NIH Image 1.37v, the SH3BP5 immunofluorescence intensity and the area of a selected neuron were quantified. Mean SH3BP5 immunofluorescence intensity per 1 μm² (a) of the neuron was calculated. Mean immunofluorescence intensity per 1 μm² of the neuropil around the neuron was simultaneously quantified as a background immunofluorescence (b). The subtracted mean immunofluorescence intensity (a−b) was used as the mean SH3BP5 immunofluorescence intensity of the neuron. Then, the a−b value was multiplied by the neuronal area to estimate the level of SH3BP5 expression of in the neuron. Ten neurons were selected at random and the mean immunofluorescence intensity in 10 neurons per sample was calculated for each sample.

### [Statistical Analysis]

All data were analyzed using Prism7 for Mac OSX software (GraphPad, San Diego, USA). Data in cell-death experiments were shown by means±standard deviation. All the other data were shown by means±SEM. Unpaired t-test (two-tailed) was used for the analysis of data obtained from histological and ELISA experiments.

TABLE 1
Data analysis of 34 autopsied subjects for the measurement of CSF adiponectin concentration

| | | | Unpaired T test (two-tailed) | | | | |
|---|---|---|---|---|---|---|---|
| | | | | | R | degrees of | P value |
| | AD | Non-AD | p | t | squared | freedom | by F test |
| n | 20 | 14 | | | | | |
| Gender M/F | 13/7 | 9/5 | | | | | |
| Age (±SEM) | 78.5 ± 0.9 | 86.3 ± 1.4 | <0.0001* | 4.92 | 0.43 | 32 | 0.20 |
| ApoE4 allele(s) % | 75.0 | 21.4 | | | | | |
| PMD (±SEM) hour | 11.7 ± 1.8 | 11.5 ± 2.1 | 0.952 | 0.061 | 0.00017 | 32 | 0.96 |
| CSF adiponectin conc. | 0.31 ± 0.13 | 0.96 ± 0.19 | 0.0065 | 2.92 | 0.21 | 32 | 0.34 |
| (±SEM) nM | | | | | | | |

PMD: postmortem duration before autopsy.
*p < 0.0001 if “more than (>)” before ages is considered to be “equal to.” See the “Age” in Table S1.

TABLE 2
Data analysis of 11 cases with ages of 81 to 88 in Table 1

| | | | Unpaired T test (two-tailed) | | | | |
|---|---|---|---|---|---|---|---|
| | | | | | R | degrees of | p value |
| | AD | Non-AD | p | t | squared | freedom | by F test |
| n | 6 | 5 | | | | | |
| Gender M/F | 2/4 | 3/2 | | | | | |
| Age (±SEM) | 83.0 ± 0.6 | 85.2 ± 1.2 | 0.106 | 1.8 | 0.264 | 9 | 0.22 |
| ApoE4 allele(s) % | 50 | 40 | | | | | |
| PMD (±SEM) hour | 10.0 ± 3.5 | 11.9 ± 2.3 | 0.668 | 0.443 | 0.021 | 9 | 0.34 |
| CSF adiponectin conc. | 0.30 ± 0.07 | 1.41 ± 0.16 | <0.0001 | 6.73 | 0.83 | 9 | 0.14 |
| (±SEM) nM | | | | | | | |

PMD: postmortem duration before autopsy.

TABLE 3
Data analysis of 13 autopsied subjects for the measurement of intraneuronal
SH3BP5 levels in outer pyramidal layers of temporal or occipital lobes

| | | | Unpaired T test (two-tailed) | | | | |
|---|---|---|---|---|---|---|---|
| | | | | | R | degrees of | P value |
| | AD | ALS | p | t | squared | freedom | by F test |
| n | 7 | 6 | | | | | |
| Gender M/F | 2/5 | 4/2 | | | | | |
| Age (±SEM) | 75.9 ± 5.1 | 66.7 ± 2.8 | 0.158 | 1.51 | 0.17 | 11 | 0.16 |
| SH3BP levels (±SEM) | 46564 ± 7737 | 79225 ± 10305 | 0.0256 | 2.56 | 0.38 | 11 | 0.62 |
| arbitrary unit | | | | | | | |

TABLE 4
Data analysis of 20 autopsied subjects for the measurement
of SH3BP5 levels in the cell lysates of temporal lobes

| | | | Unpaired T test (two-tailed) | | | | |
|---|---|---|---|---|---|---|---|
| | | | | | R | degrees of | p value |
| | AD | Non-AD | p | t | squared | freedom | by F test |
| n | 10 | 10 | | | | | |
| Gender M/F | 0/10 | 0/10 | | | | | |
| Age (±SEM) | 79.9 ± 2.9 | 79.4 ± 1.3 | 0.876* | 0.16 | 0.0020 | 13.9 | 0.032 |
| ApoE4 allele(s) % | 80 | 0 | | | | | |
| PMD (±SEM) hour | 12.7 ± 3.0 | 16.7 ± 3.1 | 0.368 | 0.92 | 0.045 | 18 | 0.95 |
| SH3BP5 levels in temporal | 103.9 ± 9.0 | 159.4 ± 16.5 | 0.0084 | 2.96 | 0.33 | 18 | 0.085 |
| lobe lysates (±SEM) | | | | | | | |
| arbitrary unit | | | | | | | |

PMD; postmortem duration before autopsy.
*p < 0.876 if “more than (>)” before ages is considered to be “equal to.” See the “Age” in Table S3.

For the analysis of ages, unpaired t-test with Welch's correction was employed because the p value was lower than 0.05 (0.032).

TABLE 5
Table S-1
Individual data of the autopsied cases for the examination of
CSF adiponectin levels

| | | | | CERAD stage or | |
|---|---|---|---|---|---|
| Age | Sex | ApoE | PMD | Diagnosis | B & B stage |
| 81 | M | 34 | 7.2 | Normal CERAD 1B | I |
| 90 | M | 33 | 7.4 | Normal CERAD 1A | II |
| 88 | M | 23 | 17.3 | Normal CERAD 1A | I |
| 86 | M | 34 | 6.1 | Normal CERAD 1A | III |
| 90 | M | 33 | 4.0 | Normal CERAD 1B | II |
| 86 | M | 33 | 16.3 | Normal CERAD 1A | I |
| >90 | M | 34 | 7.7 | Normal CERAD 1B | I |
| >90 | M | 33 | 22.3 | Normal CERAD 1A | III |
| 90 | M | 23 | 3.7 | Normal CERAD 1A | III |
| >90 | F | 23 | 5.0 | Normal CERAD 1B | II |
| 72 | F | 33 | 30.0 | Normal CERAD 1B | II |
| >90 | F | 23 | 5.2 | Normal CERAD 1B | III |
| 85 | F | 33 | 13.0 | Normal CERAD 1A | II |
| 80 | F | 33 | 16 | Normal CERAD 1A | III |
| 73 | M | 33 | 9.4 | Possible AD | III |
| 79 | M | 24 | 6.5 | Possible AD | III |

TABLE 6
Table S1-2
Individual data of the autopsied cases for
the examination of CSF adiponectin levels

| | | | | CERAD stage or | |
|---|---|---|---|---|---|
| Age | Sex | ApoE | PMD | Diagnosis | B & B stage |
| 82 | M | 34 | 1.3 | AD | V |
| 83 | M | 33 | 2.0 | AD | V |
| 73 | M | 34 | 22.3 | AD | V |
| 76 | M | 34 | 7.0 | AD | V |
| 80 | M | 44 | 6.5 | AD | V |
| 71 | M | 34 | 6.5 | AD | V |
| 78 | M | 34 | 8.0 | AD | V |
| 85 | M | 34 | 23.5 | AD | V |
| 75 | M | 34 | 12.2 | AD | V |
| 77 | M | 33 | 10.7 | AD | V |
| 83 | M | 33 | 12.8 | AD | V |
| 79 | F | 44 | 16.2 | AD | V |
| 80 | F | 33 | 16 | AD | V |
| 79 | F | 44 | 8.0 | AD | V |
| 77 | F | 44 | 35.4 | AD | V |
| 81 | F | 44 | 14.7 | AD | V |
| 84 | F | 34 | 5.9 | AD | V |
| 75 | F | 34 | 8.8 | AD | V |

ApoE: two apolipoprotein E gene alleles are shown by numbers.
PMD: postmortem duration before autopsy, B & B stage: Braak & Braak stage.
Two possible AD cases were counted as AD cases.
Mean ± SEM ages of total AD cases and non-AD cases were 78.5 ± 0.9 years old and more than 86.3 ± 1.4 years old, respectively (unpaired t-test, p < 0.0001 if “more than” before ages is considered to be “equal to”). Mean ± SEM PMDs of total AD cases and non-AD cases were 11.7 ± 1.8 hours and 11.5 ± 2.1 hours, respectively (unpaired t-test, p = 0.96).

TABLE 7
Table S2
Individual data of the autopsied cases for the
measurement of intraneuronal SH3BP5 levels in outer
pyramidal layers of temporal or occipital lobes

| | Age/Sex | CDR |
|---|---|---|
| ALS | 69/F | NE |
| | 64/F | NE |
| | 60/M | NE |
| | 79/M | NE |
| | 62/M | NE |
| | 66/M | NE |
| AD | 65/M | 3 |
| | 79/F | 3 |
| | 79/F | 3 |
| | 83/F | 3 |
| | 55/F | 3 |
| | 73/M | 3 |
| | 97/F | 3 |

Sections of outer pyramidal layers of temporal or occipital lobes were obtained from autopsied AD and ALS patients. CDR: Clinical Dementia Rating, NE: not examined.
Mean ± SEM ages of total ALS patients and AD patients were 66.7 ± 2.8 and 75.9 ± 5.1 years old, respectively (unpaired t-test, p = 0.158).

TABLE 8
Table S3
Individual data of the autopsied cases for the measurement of
SH3BP5 levels in cell lysates of temporal lobes

| | | | | CERAD stage or | |
|---|---|---|---|---|---|
| Age | Sex | ApoE | PMD | Diagnosis | B & B stage |
| >89 | F | 23 | 5.0 | Normal CERAD 1B | II |
| 78 | F | 33 | 33.0 | Normal CERAD 1A | I |
| 82 | F | 33 | 15.5 | Normal CERAD 1B | I |
| 67 | F | 33 | 8.0 | Normal CERAD 1B | I |
| 65 | F | 33 | 13.6 | Normal CERAD 1A | I |
| 72 | F | 33 | 30.0 | Normal CERAD 1B | II |
| 88 | F | 23 | 20.5 | Normal CERAD 1A | I |
| 84 | F | 33 | 23.0 | Normal CERAD 1B | I |
| >89 | F | 23 | 5.2 | Normal CERAD 1B | III |
| 85 | F | 33 | 13.0 | Normal CERAD 1A | II |
| 79 | F | 44 | 16.2 | AD | V |
| 77 | F | 34 | 6.0 | AD | V |
| 80 | F | 33 | 5.2 | AD | V |
| 79 | F | 44 | 8.0 | AD | V |
| 77 | F | 23 | 35.4 | AD | V |
| 81 | F | 44 | 14.7 | AD | V |
| 84 | F | 34 | 5.9 | AD | V |
| 75 | F | 34 | 5.9 | AD | V |
| 74 | F | 44 | 20.7 | AD | V |
| 88 | F | 34 | 8.7 | AD | V |

ApoE: two apolipoprotein E gene alleles are shown by numbers.
PMD: postmortem duration before autopsy, B & B stage: Braak & Braak stage.
Mean ± SEM ages of total AD cases and non-AD cases were 79.9 ± 2.9 years old and more than 79.4 ± 1.3 years old, respectively (unpaired t-test, p < 0.876 if “more than” before ages is considered to be “equal to”). Mean ± SEM PMDs of total AD cases and non-AD cases were 12.7 ± 3.0 hours and 16.7 ± 3.1 hours, respectively (unpaired t-test, p = 0.368).

TABLE 9
Table L1
ELISA data of CLSPCOL and wt-CLSPCOL in interstitial fluid-containing brain lysates and serum

| | | | | Abs450 nm | | | | | | |
|---|---|---|---|---|---|---|---|---|---|---|
| | | | | | | | | Del | | |
| hCLSP_N | | | Sample | 1 | 2 | 3 | Mean | Mean | Del GST | [nM] |
| 1 | Saline | | | 0.1487 | 0.1449 | 0.1412 | 0.1449 | 0.0000 | | |
| 2 | 0.15 | nM | GST-MH6G | 0.1398 | 0.1302 | 0.1202 | 0.1301 | −0.0148 | | |
| 3 | 0.3125 | nM | | 0.1267 | 0.1232 | 0.1186 | 0.1229 | −0.0221 | | |
| 4 | 0.625 | nM | | 0.1237 | 0.1246 | 0.1194 | 0.1226 | −0.0223 | | |
| 5 | 1.25 | nM | | 0.1197 | 0.1261 | 0.1205 | 0.1221 | −0.0228 | | |
| 6 | 2.5 | nM | | 0.1186 | 0.1251 | 0.1204 | 0.1214 | −0.0235 | | |
| 7 | 5 | nM | | 0.1441 | 0.1425 | 0.1322 | 0.1396 | −0.0053 | | |
| 8 | 10 | nM | | 0.1532 | 0.1629 | 0.1718 | 0.1626 | 0.0177 | | |
| 9 | 0.15 | nM | FL-MH6G-Col | 0.2195 | 0.1847 | 0.2068 | 0.2037 | 0.0588 | | |
| 10 | 0.3125 | nM | | 0.2486 | 0.2619 | 0.2724 | 0.2610 | 0.1161 | | |
| 11 | 0.625 | nM | | 0.3713 | 0.4802 | 0.5093 | 0.4536 | 0.3087 | | |
| 12 | 1.25 | nM | | 0.8920 | 0.9408 | 0.7934 | 0.8754 | 0.7305 | | |
| 13 | 2.5 | nM | | 1.7437 | 1.4888 | 1.3777 | 1.5367 | 1.3918 | | |
| 14 | 5 | nM | | 1.6351 | 1.8804 | 1.7607 | 1.7587 | 1.6138 | | |
| 15 | 10 | nM | | 2.0106 | 2.1295 | 2.1144 | 2.0848 | 1.9399 | | |
| 16 | 0.15 | nM | (1-61)-MH6G-Col | 0.3187 | 0.3336 | 0.3801 | 0.3441 | 0.1992 | | |
| 17 | 0.3125 | nM | | 0.3001 | 0.3249 | 0.3226 | 0.3158 | 0.1709 | | |
| 18 | 0.625 | nM | | 0.3292 | 0.3980 | 0.3825 | 0.3699 | 0.2250 | | |
| 19 | 1.25 | nM | | 0.3365 | 0.3472 | 0.3428 | 0.3422 | 0.1973 | | |
| 20 | 2.5 | nM | | 0.8118 | 0.6822 | 0.6969 | 0.7303 | 0.5854 | | |
| 21 | 5 | nM | | 1.2976 | 1.4922 | 1.2312 | 1.3403 | 1.1954 | | |
| 22 | 10 | nM | | 1.8819 | 1.6769 | 1.9687 | 1.8425 | 1.6976 | | |

| | | | | | | | | |
|---|---|---|---|---|---|---|---|---|
| 23 | x10 ISF lysate 10 nmol GST-MH6G i.p. | 0.5537 | 0.5755 | 0.6151 | 0.5815 | 0.4365 | 0.0000 | 0.0000 |
| 24 | x10 ISF lysate 10 nmol FL-MH6G-Col i.p. | 0.5214 | 0.6803 | 0.5786 | 0.5934 | 0.4485 | 0.0120 | 0.0701 |
| 25 | x10 ISF lysate 10 nmol (1-61)-MH6G-Col i.p. | 1.0356 | 1.0958 | 1.1772 | 1.1029 | 0.9580 | 0.5214 | 2.4095 |
| 26 | x50 serum 10 nmol GST-MH6G i.p. | 0.1240 | 0.1305 | 0.1614 | 0.1386 | −0.0063 | 0.0000 | 0.0000 |
| 27 | x50 serum 10 nmol FL-MH6G-Col i.p. | 0.6937 | 0.6669 | 0.7998 | 0.7201 | 0.5752 | 0.5815 | 1.0628 |
| 28 | x50 serum 10 nmol (1-61)-MH6G-Col i.p. | 1.2742 | 1.3508 | 1.3185 | 1.3145 | 1.1696 | 1.1759 | 6.1049 |

Stepwise increasing concentrations of recombinant protein were measured to create a standard dose-response line (N=3). To measure the concentrations of CLSPCOL or wt-CLSPCOL in ISF-containing brain lysates and serum, 90 μL of PBS containing 10 μL of interstitial fluid-containing brain lysates (×10 ISF lysate) or 98 μL of PBS containing 2 μL of serum (serum ×50) were subjected to ELISA (N=3). The values obtained by actually measuring stepwise increasing concentrations of standard recombinant proteins (GST-MycHisG, wt-CLSPCOL, and CLSPCOL; concentration: 0.15 to 10 nM), ×10 ISF lysates, and ×50 serum were shown in Abs450 columns. Subsequently, the mean of 3 values was calculated and shown in Mean Abs450 columns. The mean value of saline was subtracted from each mean value to obtain the Del Mean value. The Del GST values of ×10 ISF lysates and ×50 serum were obtained by subtracting the Del Mean value of GST-MycHisG (negative control) from the Del Mean value of CLSPCOL or wt-CLSPCOL. The concentrations of recombinant proteins in ISF-containing brain lysates and serum were then estimated using standard dose-response lines (FIG. 6a).

### INDUSTRIAL APPLICABILITY

The CLSP derivative, polypeptide, potentiator or protector, and fusion protein involved in the present invention are useful as an active ingredient of a pharmaceutical composition to suppress Alzheimer's disease-related neuronal cell dysfunction or neuronal cell death, for example a pharmaceutical composition used to prevent or treat diseases accompanied by Alzheimer's disease-related memory impairment or neurodegeneration.

The list of literature cited in the description is described below.

### LIST OF CITED LITERATURE

1. Selkoe, D. J. SnapShot: pathobiology of Alzheimer's disease. Cell 154, 468-468 (2013).

2. Selkoe, D. J., & Hardy, J. The amyloid hypothesis of Alzheimer's disease at 25 years. EMBO Mol. Med. 8, 595-608. (2016).

3. Scheltens, P. Alzheimer's disease. Lancet, 388, 505-517 (2016).

4. Hashimoto, Y, Kurita, M., Aiso, S., Nishimoto, I., & Matsuoka, M. Humanin inhibits neuronal cell death by interacting with a cytokine receptor complex or complexes involving CNTF receptor a/WSX-1/gp130. Mol. Biol. Cell 20, 2864-2873 (2009).

5. Hashimoto, Y. et al. Secreted calmodulin-like skin protein inhibits neuronal cell death in cell-based Alzheimer's disease models via the heterotrimeric Humanin receptor. Cell Death Dis. 4:e555 (2013).

6. Matsuoka, M. Protective effects of Humanin and calmodulin-like skin protein in Alzheimer's disease and broad range of abnormalities. Mol. Neurobiol. 51, 1232-1239 (2015).
7. Matsuoka, M. HUMANIN: A Defender against Alzheimer's disease. Recent Patents CNS Drug Discov. 4, 37-42 (2009).
8. Kusakari, S., Nawa, M., Sudo, K., & Matsuoka, M. Calmodulin-like skin protein protects against spatial learning impairment in a mouse model of Alzheimer's disease. J. Neurochem. 144, 218-233 (2018).
9. Widmer, R. J. et al. Circulating humanin levels are associated with preserved coronary endothelial function. Am. J. Physiol. Heart Circ. Physiol. 304, H393-7 (2013).
10. Mehul, B., Bernard, D., Simonetti, L., Bernard, M. A., & Schmidt, R. Identification and cloning of a new calmodulin-like protein from human epidermis. J. Biol. Chem. 275, 12841-12847 (2000).
11. Hwang, M., & Morasso, M. I. The novel murine Ca2+-binding protein, Scarf, is differentially expressed during epidermal differentiation. J. Biol. Chem. 278, 47827-47833 (2003).
12. Hwang, M., Kalinin, A., & Morasso, M. I. The temporal and spatial expression of the novel Ca++-binding proteins, Scarf and Scarf2, during development and epidermal differentiation. Gene Expr. Patterns 5, 801-808 (2005).
13. Hayashi, M., Tajima, H., Hashimoto, Y, & Matsuoka, M. Secreted calmodulin-like skin protein ameliorates scopolamine-induced memory impairment. Neuroreport 25, 725-729 (2014).
14. Hashimoto, Y, Umahara, T., Hanyu, H., Iwamoto, T., & Matsuoka, M. Calmodulin-like skin protein is downregulated in human cerebrospinal fluids of Alzheimer's disease patients with apolipoprotein E4; a pilot study using postmortem samples. Neurol. Res. 39, 767-772 (2017).
15. Hwang, J. et al. Role of Scarf and its binding target proteins in epidermal calcium homeostasis. J. Biol. Chem. 282, 18645-18653 (2007).
16. Rebeck, G. W. The role of APOE on lipid homeostasis and inflammation in normal brains. J. Lipid Res. 58, 1493-1499 (2017).
17. Liao, F., Yoon, H., & Kim, J. Apolipoprotein E metabolism and functions in brain and its role in Alzheimer's disease. Curr. Opin. Lipidol. 28, 60-67 (2017)
18. Lindh, M. et al. Cerebrospinal fluid apolipoprotein E (apoE) levels in Alzheimer's disease patients are increased at follow up and show a correlation with levels of tau protein. Neurosci Lett. 229, 85-88 (1997).
19. Schmidt, C. et al. Cerebrospinal fluid apolipoprotein E concentration and progression of Alzheimer's disease. J. Alzheimers Dis. 43, 1229-1236 (2015).
20. Wahrle, S. E. et al. Apolipoprotein E levels in cerebrospinal fluid and the effects of ABCAlpolymorphisms Mol. Neurodegener. 2, 7 (2007).
21. Matsui, Y. et al. High sensitivity of an ELISA kit for detection of the gamma-isoform of 14-3-3 proteins: usefulness in laboratory diagnosis of human prion disease. BMC Neurol. 11, 120 (2011).
22. Liu, R. et al. Calreticulin as a potential diagnostic biomarker for lung cancer. Cancer Immunol. Immunother. 61, 855-864 (2012).
23. Liu, M., & Liu, F. Transcriptional and post-translational regulation of adiponectin Biochem. J. 425, 41-52 (2010).
24. Takeshita, Y, Hashimoto, Y., Nawa, M., Uchino, H. & Matsuoka, M. SH3-binding protein 5 mediates the neuroprotective effect of the secreted bioactive peptide humanin by inhibiting c-Jun NH2-terminal kinase. J.Biol. Chem. 288, 24691-24704 (2013).
25. Niikura, T., Sidahmed, E., Hirata-Fukae, C., Aisen, P. S., Matsuoka, Y. A humanin derivative reduces amyloid beta accumulation and ameliorates memory deficit in triple transgenic mice. PloS one 6:e16259 (2011).
26. Zhang, W. et al. S14G-humanin improves cognitive deficits and reduces amyloid pathology in the middle-aged APPswe/PS1dE9 mice. Pharmacol. Biochem. Behavior 100, 361-369. (2012).
27. Yin, R. et al. Protective Effects of Colivelin Against Alzheimer's disease in a PDAPP Mouse Model. Cell. physiol. Biochem. 38, 1138-1146 (2016).
28. Combs T. P., Berg A. H., Obici S., Scherer P. E., & Rossetti L. Endogenous glucose production is inhibited by the adipose-derived protein Acrp30. J. Clin. Investig. 108, 1875-1881 (2001).
29. Une, K.et al. Adiponectin in plasma and cerebrospinal fluid in MCI and Alzheimer's disease. Eur. J. Neurol. 18, 1006-1009 (2011).
30. Waragai, M. et al. Possible Involvement of Adiponectin, the Anti-Diabetes Molecule, in the Pathogenesis of Alzheimer's disease. J. Alzheimers Dis. 52, 1453-9 (2016).
31. Ng, R. C., & Chan, K. H. Potential Neuroprotective Effects of Adiponectin in Alzheimer's disease. Int. J. Mol. Sci. 18, pii: E592 (2017).
32. Himbergen, T. M. V. et al. Biomarkers for Insulin Resistance and Inflammation and the Risk for All-Cause Dementia and Alzheimer Disease Results From the Framingham Heart Study. Arch. Neurol. 69, 564-600 (2012).
33.Garcia-Casares N. et al. Alzheimer's like brain changes correlate with low adiponectin plasma levels in type 2 diabetic patients. J. Diabetes Complicat. 30, 281-286 (2016).
34. Ng, R. C. L. et al. Chronic adiponectin deficiency leads to Alzheimer's disease-like cognitive impairments through AMPK inactivation and cerebral insulin resistance in aged mice. Mol. Neurodegener. 11,71 (2016).
35. Chiba, T. et al. Amyloid-beta causes memory impairment by disturbing the JAK2/STAT3 axis in hippocampal neurons. Mol. Psychiatry 14, 206-222 (2009).
36. Liu, X. et al. Unbound drug concentration in brain homogenate and cerebral spinal fluid at steady state as a surrogate for unbound concentration in brain interstitial fluid. Drug Metab. Dispos. 37, 787-93 (2009).
37. Sweeney, M. D., Sagare, A. P., & Zlokovic, B. V. Blood-brain barrier breakdown in Alzheimer disease and other neurodegenerative disorders. Nat. Rev. Neurol. 14, 133-150 (2018).
38. Liu, C.-C., Kanekiyo, T., Xu, H., & Bu, G. Apolipoprotein E and Alzheimer disease: risk, mechanisms, and therapy Nat. Rev. Neurol. 9, 106-118 (2012).
39. Yamatsuji, T., et al. G protein-mediated neuronal DNA fragmentation induced by familial Alzheimer's disease-associated mutants of APP. Science 272, 1349-1352 (1996).
40. Mirra, S. S. et al. The Consortium to Establish a Registry for Alzheimer's Disease (CERAD). Part II. Standardization of the neuropathologic assessment of Alzheimer's disease. Neurology 41, 479-486 (1999).
41. Braak H., & Braak E. Neuropathological staging of Alzheimer-related changes. Acta Neuropathol (Berl) 82, 239-59 (1991).
42. Pajvani, U. B. et al. Structure-function studies of the adipocyte-secreted hormone Acrp30/adiponectin. Implications for metabolic regulation and bioactivity J. Biol. Chem. 278, 9073 (2003)

1. A derivative (mutant) of calmodulin-like skin protein (CLSP), comprising an endogenous humanin-homogenous region (EHR), which is a core of an activity to suppress Alzheimer's disease-related neuronal cell dysfunction or neuronal cell death (CLSP activity), and not comprising a region to which an inhibitor of the CLSP activity binds.

2. The derivative according to claim 1, wherein EHR consists of an amino acid sequence (I): TGKNLSEAQLRKLISEVDS(or “G”)DGD (amino acid single letter code) (I).

3. The derivative according to claim 1, wherein the region to which the inhibitor binds is a C-terminal amino acid sequence region (amino acid 62-146) of CLSP (SEQ ID NO:1).

4. The derivative according to claim 1, which is a polypeptide consisting of an amino acid sequence below:
(1) an N-terminal amino acid sequence region (amino acid 1-61) of CLSP;
(2) an amino acid sequence of the (1) above, wherein one or several (e.g., about 2-5) amino acids are deleted, substituted or inserted in an amino acid sequence other than EHR included in the amino acid sequence of the (1) above; or
(3) an amino acid sequence of the (1) above, which has an identity of 90% or more, preferably 95% or more, and further preferably 98% or more to an amino acid sequence other than EHR included in the amino acid sequence of the (1) above.

5. The derivative according to claim 1, which is insensitive to an inhibitory or suppressive action by the inhibitor of the CLSP activity.

6. The derivative according to claim 1, wherein the inhibitor is selected from a group consisting of apolipoprotein E, 14-3-3 proteins, and calreticulin.

7. A polypeptide consisting of an amino acid sequence below:
(1) an amino acid sequence (ADNCol) shown by SEQ ID NO:2;
(2) an amino acid sequence comprising the amino acid sequence (ADNCol) of the (1) above;
(3) an amino acid sequence of adiponectin shown by SEQ ID NO:3, wherein one or several amino acids are deleted, substituted or inserted in an amino acid sequence other than ADNCol included in the amino acid sequence shown by SEQ ID NO:3; or
(4) an amino acid sequence of adiponectin shown by SEQ ID NO:3, which has an identity of 90% or more to an amino acid sequence other than ADNCol included in the amino acid sequence shown by SEQ ID NO:3.

8. A potentiator or protector of the CLSP activity by CLSP or a CLSP derivative, which consists of a polypeptide according to claim 7, wherein the CLSP derivative comprises an endogenous humanin-homogenous region (EHR), which is a core of an activity to suppress Alzheimer's disease-related neuronal cell dysfunction or neuronal cell death (CLSP activity) and does not comprise a region to which an inhibitor of the CLSP activity binds.

9. The potentiator or protector according to claim 8, which protects the CLSP from the inhibitory or suppressive action by the inhibitor of the CLSP activity, or nullifies the action by the inhibitor.

10. The potentiator or protector according to claim 8, wherein the polypeptide is adiponectin.

11. The potentiator or protector according to claim 8, wherein the inhibitor is selected from a group consisting of apolipoprotein E, 14-3-3 proteins, and calreticulin.

12. A fusion protein comprising CLSP or a CLSP derivative and the polypeptide according claim 7, wherein the CLSP derivative comprises an endogenous humanin-homogenous region (EHR), which is a core of an activity to suppress Alzheimer's disease-related neuronal cell dysfunction or neuronal cell death (CLSP activity) and does not comprise a region to which an inhibitor of the CLSP activity binds.

13. The fusion protein according to claim 12, consisting of the N-terminal amino acid sequence region (amino acid 1-61) of CLSP, and ADNCol.

14. The fusion protein according to claim 12, which is insensitive to the inhibitory or suppressive action by the inhibitor of the CLSP activity.

15. A pharmaceutical composition to suppress Alzheimer's disease-related neuronal cell dysfunction or neuronal cell death, the composition comprising as an active ingredient the fusion protein according to claim 12.

16. The pharmaceutical composition according to claim 15, which is used to prevent or treat diseases accompanied by Alzheimer's disease-related memory impairment or neurodegeneration.

17. A method for treating a disorder accompanied by neuronal cell dysfunction or neuronal cell death, or a disease accompanied by memory impairment or neurodegeneration, the method comprising a stage of administering the pharmaceutical composition according to claim 15 to an individual affected with or suspected of the disorder or the disease.

18. The method according to claim 17, wherein the disorder or disease is Alzheimer's disease.

19. A method for detecting an activity to suppress Alzheimer's disease-related neuronal cell dysfunction or neuronal cell death by the fusion protein according to claim 12 (the polypeptide of the invention), the method comprising (a) a step of inducing the neuronal cell dysfunction or neuronal cell death in a presence/absence of the CLSP inhibitor, and in a presence/absence of the polypeptide of the invention, (b) a step of detecting the neuronal cell dysfunction or neuronal cell death, and (c) a step of comparing the neuronal cell dysfunction or neuronal cell death in the presence/absence of the polypeptide of the invention.

20. A method for screening a substance regulating an activity to suppress Alzheimer's disease-related neuronal cell dysfunction or neuronal cell death by the fusion protein according to claim 12 (the polypeptide of the invention) or CLSP,
the method comprising (a) a step of inducing the neuronal cell dysfunction or neuronal cell death with or without a test substance in the presence of the polypeptide of the invention or CLSP, (b) a step of detecting the neuronal cell dysfunction or neuronal cell death, and (c) a step of selecting a substance regulating the activity to suppress the neuronal cell dysfunction or neuronal cell death by the polypeptide of the invention or CLSP.


SEQUENCE LISTING (11 sequences)

SEQ ID NO: 1 - protein, 146 aa, Homo sapiens
  CLSP
MAGELTPEEEAQYKKAFSAVDTDGNGTINAQELGAALKATGKNLSEAQLRKLISEVDSDGDGEISFQEFLTAAKKARAGLEDLQVAFRAFDQDGDGHITVDELRRAMAGLGQPLPQEELDAMIREADVDQDGRVNYEEFARMLAQE

SEQ ID NO: 2 - protein, 60 aa, Artificial Sequence
  ADNCol
GHPGHNGAPGRDGRDGTPGEKGEKGDPGLIGPKGDIGETGVPGAEGPRGFPGIQGRKGEP

SEQ ID NO: 3 - protein, 244 aa, Homo sapiens
  Adiponectin (ADN)
MLLLGAVLLLLALPGHDQETTTQGPGVLLPLPKGACTGWMAGIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 4 - DNA, 48 bp, Artificial Sequence
  sense sequence for HiBit
cccggggtgagcggctggcggctgttcaagaagattagctgagaattc

SEQ ID NO: 5 - DNA, 48 bp, Artificial Sequence; identical to SEQ ID NO: 4
  anti-sense sequence for HiBit

SEQ ID NO: 6 - DNA, 43 bp, Artificial Sequence
  sense primer
ggttgagaattcatcgtgactgactgacgatctgcctcgcgcg

SEQ ID NO: 7 - DNA, 42 bp, Artificial Sequence
  anti-sense primer
atgatgatgatgatgatgatcctcttctgagatgagtttttg

SEQ ID NO: 8 - DNA, 42 bp, Artificial Sequence
  sense primer
ggatccatgagaggatcgcatcaccatcaccatcacgggtcc

SEQ ID NO: 9 - DNA, 44 bp, Artificial Sequence
  anti-sense primer
gaattctcaaggttctcctttcctgccttggattcccggaaagc

SEQ ID NO: 10 - DNA, 88 bp, Artificial Sequence
  sense primer
aagcttgaacaaaaactcatctcagaagaggatcatcatcatcatcatcatggtatggggcatccgggccataatggggccccaggcc

SEQ ID NO: 11 - DNA, 45 bp, Artificial Sequence
  anti-sense primer
gaattctcaaggttctcctttcctgccttggattcccggaaagcc